(19) United States
(12) Patent Application Publication
(10) Pub. No.: US 2007/0010562 A1
(43) Pub. Date: Jan. 11, 2007

(54) Nr1h4 nuclear receptor binding compounds

(76) Inventors: Ulrike Bauer, Sandhausen (DE); Zach Cheruvallath, San Diego, CA (US); Ulrich Deuschle, Bammental (DE); Elena Dneprovskaia, San Diego, CA (US); Tim Gahman, Encinitas, CA (US); Kristina Giegrich, Lampetheim (DE); Ronnie Hanecak, San Clemente, CA (US); Normand Hebert, Cardiff, CA (US); John Kiely, San Diego, CA (US); Ingo Kober, Gaiberg (DE); Manfred Kogl, Eppelheim (DE); Harald Kranz, Leimen (DE); Claus Kremoser, Heidelberg (DE); Matthew Lee, Solana Beach, CA (US); Kerstin Otte, Heidelberg (DE); Carlton Sage, Cardiff, CA (US); Manish Sud, San Diego, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/486,748

(22) PCT Filed: Aug. 13, 2002
(86) PCT No.: PCT/EP02/09076

§ 371 Date: Sep. 20, 2004

Related U.S. Application Data
(63) Continuation-in-part of application No. 10/185,721, filed on Jul. 1, 2002, now Pat. No. 7,034,046.
Application No. 10/185,721 is a continuation-in-part of application No. 10/185,731, filed on Jul. 1, 2002, now Pat. No. 6,974,830.

(30) Foreign Application Priority Data
Aug. 31, 2001 (EP) 01119473.5

(51) Int. Cl. A61K 31/4436; A61K 31/44; C07D 409/04; C07D 213/72

(52) U.S. Cl. 514/342; 514/352; 546/280.4; 546/304

(57) ABSTRACT

The present invention relates to compounds according to the general formula (1) which bind to the NR1H4 receptor and act as agonists, antagonists or mixed agonists/antagonists of the NR1H4 receptor. The invention further relates to the treatment of diseases and/or conditions through binding of said nuclear receptor by said compounds and the production of medicaments using said compounds.

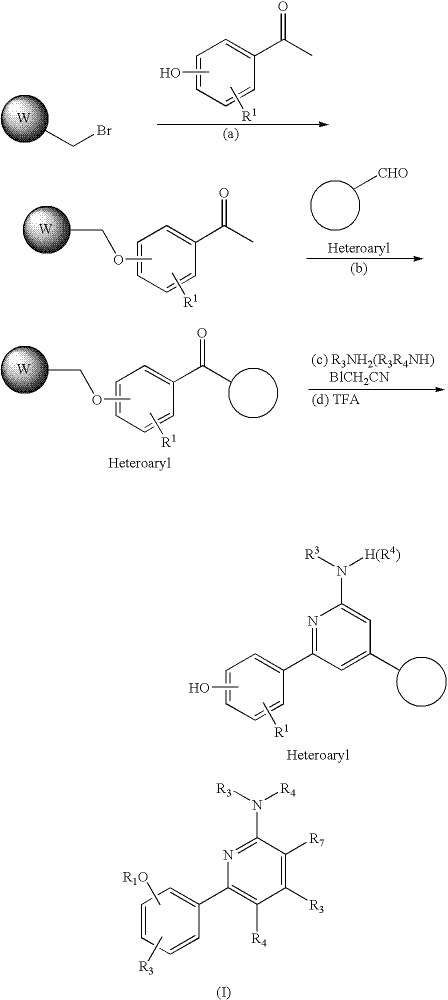

### BACKGROUND OF THE INVENTION

Multicellular organisms are dependent on advanced mechanisms of information transfer between cells and body compartments. The information that is transmitted can be highly complex and can result in the alteration of genetic programs involved in cellular differentiation, proliferation, or reproduction. The signals, or hormones, are often simple molecules, such as peptides, fatty acid, or cholesterol derivatives.

Many of these signals produce their effects by ultimately changing the transcription of specific genes. One well-studied group of proteins that mediate a cells response to a variety of signals is the family of transcription factors known as nuclear receptors, hereinafter referred to often as “NR”. Members of this group include receptors for steroid hormones, vitamin D, ecdysone, cis and trans retinoic acid, thyroid hormone, bile acids, cholesterol-derivatives, fatty acids (and other peroxisomal proliferators), as well as so-called orphan receptors, proteins that are structurally similar to other members of this group, but for which no ligands are known (Escriva, H. et al., Ligand binding was acquired during evolution of nuclear receptors, PNAS, 94, 6803-6808, 1997). Orphan receptors may be indicative of unknown signaling pathways in the cell or may be nuclear receptors that function without ligand activation. The activation of transcription by some of these orphan receptors may occur in the absence of an exogenous ligand and/or through signal transduction pathways originating from the cell surface (Mangelsdorf, D. J. et al., The nuclear receptor superfamily: the second decade, Cell 83, 835-839, 1995).

In general, three functional domains have been defined in NRs. An amino terminal domain is believed to have some regulatory function. A DNA-binding domain hereinafter referred to as “DBD” usually comprises two zinc finger elements and recognizes a specific Hormone Responsive Element hereinafter referred to as “HRE” within the promoters of responsive genes. Specific amino acid residues in the “DBD” have been shown to confer DNA sequence binding specificity (Schena, M. & Yamamoto, K. R., Mammalian Glucocorticoid Receptor Derivatives Enhance Transcription in Yeast, Science, 241:965-967, 1988). A Ligand-binding-domain hereinafter referred to as “LBD” is at the carboxy-terminal region of known NRs. In the absence of hormone, the LBD of some but not all NRs appears to interfere with the interaction of the DBD with its HRE. Hormone binding seems to result in a conformational change in the NR and thus opens this interference (Brzozowski et al., Molecular basis of agonism and antagonism in the oestrogen receptor, Nature, 389, 753-758, 1997; Wagner et al., A structural role for hormone in the thyroid hormone receptor, Nature, 378, 690-697.1995). A NR without the HBD constitutively activates transcription but at a low level.

Coactivators or transcriptional activators are proposed to bridge between sequence specific transcription factors and the basal transcription machinery and in addition to influence the chromatin structure of a target cell. Several proteins like SRC-1, ACTR, and Grip1 interact with NRs in a ligand enhanced manner (Heery et al., A signature motif in transcriptional coactivators mediates binding to nuclear receptors, Nature, 387, 733-736; Heinzel et al., A complex containing N-CoR, mSin3 and histone deacetylase mediates transcriptional repression, Nature 387, 43-47, 1997). Furthermore, the physical interaction with repressing receptor-interacting proteins or corepressors has been demonstrated (Xu et al., Coactivator and Corepressor complexes in nuclear receptor function, Curr Opin Genet Dev, 9 (2), 140-147, 1999).

Nuclear receptor modulators like steroid hormones affect the growth and function of specific cells by binding to intracellular receptors and forming nuclear receptor-ligand complexes. Nuclear receptor-hormone complexes then interact with a hormone response element (HRE) in the control region of specific genes and alter specific gene expression.

The Farnesoid X Receptor alpha (FXR; hereinafter also often referred to as NR1H4 when referring to the human receptor) is a prototypical type 2 nuclear receptor which activates genes upon binding to promoter region of target genes in a heterodimeric fashion with Retinoid X Receptor (hereinafter RXR, Forman et al., Cell, 81, 687-93, 1995). The relevant physiological ligands of NR1H4 seem to be bile acids (Makishima et al., Science, 284, 1362-65, 1999; Parks et al., Science, 284, 1365-68, 1999). The most potent is chenodeoxycholic acid, which regulates the expression of several genes that participate in bile acid homeostasis. Farnesol, originally described to activate the rat ortholog at high concentration does not activate the human or mouse receptor. FXR is expressed In the liver, small intestine, colon, ovary, adrenal gland and kidney. Like LXR-α, NR1H4 is involved in autocrine signaling.

FXR is proposed to be a nuclear bile acid sensor. As a result, it modulates both, the synthetic output of bile acids from the liver and their recycling in the intestine (by regulating bile acid binding protein). Upon activation (e.g. binding of chenodeoxycholic acid) it influences the conversion of dietary cholesterol into bile acids by by inhibiting the transcription of key genes which are involved in bile acid synthesis such as CYP7A1 or in bile acid transport across the hepatocyte membranes such as the bile acid transporters BSEP (=Bile Salt Export Pump) and NTCP (Na-Taurocholate Co-Transporter). This seems to be a major mechanism of feedback regulation onto bile acid synthesis. Moreover, NR1H4 seems to be the crucial receptor for maintaining bile acid homeostasis within the hepatocyte and therefore might be an appropriate drug target to treat diseases that result from impaired bile acid production, impaired export into the bile canaliculi or impaired bile flow in general such as cholestatic conditions. Loss of function of NR1H4 results in major changes in bile acid homeostasis on the organism level (Lu, et al., Mol Cell. (2000) 6(3):507-15; Goodwin, et al., Mol Cell. (2000) 6(3):517-26; Sinai, et al., Cell (2000) 15;102(6):731-44).

The synthetic compounds, 1,1-bisphosphonate esters, appear to display a number of similar activities to the two identified prototypes of natural FXR agonists, farnesol, and chenodeoxycholic acid. Like farnesol, the 1,1- bisphosphonate esters increase the rate of 3-Hydroxy-3-methylglutaryl-CoA (HMG-CoA) Reductase degradation and like bile acids they induce the expression of the Intestinal Bile Acid Binding Protein (I-BABP) and repress the cholesterol 7 α-hydroxylase gene. Certain 1,1-bisphosphonate esters also bind to FXR. (Niesor et al., Curr Pharm Des,7(4):231-59, 2001). That means that activation of FXR could lead to opposing effects (lowering the rate of cholesterol synthesis by increasing degradation of HMG-CoA Reductase and increasing the cholesterol pool by inhibition of cholesterol degradation into bile acids). The FXR agonist chenodeoxycholic acid does not change cholesterol and lipoprotein levels significantly in patients, although a repression of bile acid synthesis as well as a decreased HMG-CoA Reductase activity was observed (Einarsson et al., Hepatology, 33(5), 1189-93, 2001) confirming that cellular cholesterol synthesis and degradation are controlled by numerous regulatory loops including the coordinate regulation of HMGCoA reductase and cholesterol 7α-hydroxylase and that compounds modulating FXR acitvity might have different effects on blood lipid parameters.

In the course of functional analysis of certain 1,1-bisphosphonate esters, it was shown that these compounds which are known to bind to FXR also induce apoptosis in a variety of cell types, similar to the isoprenoids farnesol and geranylgeraniol which are also known as weak FXR binders (Flach et al., Biochem Biophys Res Com, 270, 240-46, 2000).

To date only very few compounds have been described which bind the NR1H4 receptor and thus show utility for treating diseases or conditions which are due to or influenced by said nuclear receptor (Maloney at al., J Med Chem, 10; 43(16):2971-4, 2000).

It is currently believed that FXR agonists might be useful to treat cholestatic conditions because they result in an upregulation of bile acid transport activity across the canalicular hepatocyte membrane (Plass, et al., Hepatology. (2002) 35(3):589-96; Willson, et al., Med Res Rev. (2001) 21(6):513-22). In contrast, it is believed that compounds that act as FXR antagonists or at least as mixed agonists/antagonists might reduce total serum cholesterol (Urizar, et al., Science (2002) 31;296(5573):1703-6).

It was thus an object of the present invention to provide for novel NR1H4 binding compounds. It was thus an object of the present invention to provide for compounds which by means of binding the NR1H4 receptor act as agonist or antagonist or mixed agonist/antagonist of said receptor and thus show utility for treating diseases or conditions which are due to or influenced by said nuclear receptor.

It was further an object of the invention to provide for compounds which may be used for the manufacture of a medicament for the treatment of cholesterol or bile acid associated conditions or diseases. In a preferred embodiment of the invention it was an object of the invention to provide for cholesterol lowering or anti-cholestatic compounds. It was also an object of the invention to provide for compounds that may be used for the manufacture of anticancer medicaments or apoptosis-inducing medicaments in general.

It was further an object of the invention to provide for compounds which are orally available and can be used for an oral treatment of the diseases mentioned afore.

### SUMMARY OF THE INVENTION

The present invention provides, inter alia, novel NR1H4 nuclear receptor protein binding compounds according to the general formulae (1), (2), (3), (4) shown below. Said compounds are also binders of mammalian homologues of said receptor. Further the object of the invention was solved by providing for amongst the NR1H4 nuclear receptor protein binding compounds according to the general formulae (1), (2), (3), (4) such compounds which act as agonists and such compounds which act as antagonists or mixed agonists/antagonists of the human FXR receptor or a mammalian homologue thereof.

The invention provides for FXR agonists which may be used for the manufacture of a medicament for the treatment of cholesterol or bile acid associated conditions or diseases or for the treatment of hyperproliferative diseases such as cancer or for the treatment of drug resistance which results from continous drug treatment of cancer or infectious diseases. In a preferred embodiment of the invention it was an object of the invention to provide for cholesterol lowering or anti-cholestatic compounds. It was also an object of the invention to provide for compounds that may be used for the manufacture of anticancer medicaments or apoptosis-inducing medicaments in general.

The foregoing merely summarizes certain aspects of the present invention and is not intended, nor should it be construed, to limit the invention in any manner. All patents and other publications recited herein are hereby incorporated by reference in their entirety.

### DETAILED DESCRIPTION OF THE PREFERRED EMBODIMENTS

The invention provides for a compound of the formula (1), or pharmaceutical acceptable salts or solvates thereof, hereinafter also referred to as the “compounds according to the invention” including particular and preferred embodiments thereof. 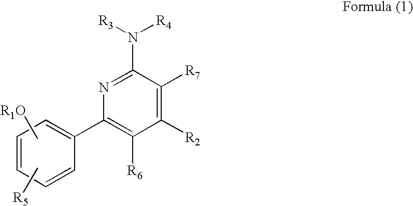
Wherein in formula (1) as shown above, R₁ is H, C₁ to C₇ acyl or C₁ to C₇ substituted acyl, R₂ is phenyl, substituted phenyl, C₅ to C₆ heteroaryl, C₅ to C₆ substituted heteroaryl, naphthyl or substituted naphthyl, R₃ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl, C₇ to C₁₂ substituted phenylalkyl, or phenyl,
R₄ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl or C₇ to C₁₂ substituted phenylalkyl,
R₃ and R₄ may be taken together with nitrogen to form a heterocycle or substituted heterocycle, or a heteroaryl or substituted heteroaryl ring, R₅ is H, C₁ to C₈ alkyl, halogen, C₁ to C₈ alkoxy, carboxy, ester, amide, susbstituted amide or C₁ to C₈ aminoacyl, R₆ is H, C₁ to C₈ alkyl, C₁ to C₈ substituted alkyl and R₇ is H C₁ to C₈ alkyl, C₁ to C₈ sustituted alkyl, halogen, C₁ to C₈ alkoxyl, C₁ to C₈ susbstituted alkoxyl.

The inventors have unexpectedly identified the compounds as well as the general structure capable of effectively binding FXR and as claimed in the present invention amongst approximately 16280 compounds that were within a compound library as disclosed in WO 01/23887 entitled “2-aminopyridine derivatives and combinatorial libraries thereof”.

The compounds of the invention can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

The term “halogen” refers to the fluoro, chloro, bromo or iodo atoms. There can be one or more halogen, which are the same or different. Preferred halogens are chloro and fluoro.

The symbol “H” denotes a hydrogen atom.

The term “C₁ to C₇ acyl” encompasses groups such as formyl, acetyl, propionyl, butyryl, pentanoyl, pivaloyl, hexanoyl, heptanoyl, benzoyl and the like. Preferred acyl groups are acetyl and benzoyl.

The term “C₁ to C₇ substituted acyl” denotes the acyl group substituted by one or more, and preferably one or two, halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, C₁ to C₆ alkyl ester, carboxy, protected carboxy, carbamoyl, carboxamide, protected carboxamide, N—(C₁ to C₆ alkyl)carboxamide, protected N—(C₁ to C₆ alkyl)carboxamide, N,N-di(C₁ to C₆ alkyl)carboxamide, cyano, methylsulfonylamino, thiol, C₁ to C₄ alkylthio or C₁ to C₄ alkylsulfonyl groups. The substituted acyl groups may be substituted once or more, and preferably once or twice, with the same or with different substituents.

Examples of C₁ to C₇ substituted acyl groups include 4-phenylbutyroyl, 3-phenylbutyroyl, 3-phenylpropanoyl, 2-cyclohexanylacetyl, cyclohexanecarbonyl, 2-furanoyl and 3-dimethylaminobenzoyl and the like.

The term “substituted phenyl” specifies a phenyl group substituted with one or more, and preferably one or two, moieties chosen from the groups consisting of halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, wherein the phenyl is substituted or unsubstituted, such that, for example, a biphenyl results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3 or 4-hydroxyphenyl, 2,4-dihydroxyphenyl, the protected-hydroxy derivatives thereof and the like; a nitrophenyl group such as 2, 3 or 4-nitrophenyl; a cyanophenyl group, for example, 2, 3 or 4-cyanophenyl; a mono- or di(alkyl)phenyl group such as 2, 3 or 4-methylphenyl, 2,4-dimethylphenyl, 2, 3 or 4-(iso-propyl)phenyl, 2, 3 or 4-ethylphenyl, 2, 3 or 4-(n-propyl)phenyl and the like; a mono or di(alkoxyl)phenyl group, for example, 2,6-dimethoxyphenyl, 2, 3 or 4-methoxyphenyl, 2, 3 or 4-ethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono-or di(hydroxymethyl)phenyl or (protected hydroxymethyl)phenyl such as 2,3, or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy 4-chlorophenyl and the like.

The term “heteroaryl” means a heterocyclic aromatic derivative which is a five-membered or six-membered ring system having from 1 to 4 heteroatoms, such as oxygen, sulfur and/or nitrogen, in particular nitrogen, either alone or in conjunction with sulfur or oxygen ring atoms. Examples of heteroaryls include pyridinyl, pyrimidinyl, and pyrazinyl, pyridazinyl, pyrrolo, furano, thiopheno, oxazolo, isoxazolo, phthalimido, thiazolo and the like.

The term “substituted heteroaryl” means the above-described heteroaryl is substituted with, for example, one or more, and preferably one or two, substituents which are the same or different which substituents can be halogen, hydroxy, protected hydroxy, cyano, nitro, C₁ to C₆ alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino or N-(phenylsulfonyl)amino groups.

The term “substituted naphthyl” specifies a naphthyl group substituted with one or more, and preferably one or two, moieties either on the same ring or on different rings chosen from the groups consisting of halogen, hydroxy, protected hydroxy, cyano, nitro, C₁ to C₆ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino or N-(phenylsulfonyl)amino.

Examples of the term “substituted naphthyl” includes a mono or di(halo)naphthyl group such as 1, 2, 3, 4, 5, 6, 7 or 8-chloronaphthyl, 2,6-dichloronaphthyl, 2,5-dichloronaphthyl, 3,4-dichloronaphthyl, 1, 2, 3, 4, 5, 6, 7 or 8-bromonaphthyl, 3,4-dibromonaphthyl, 3-chloro-4-fluoronaphthyl, 1, 2, 3, 4, 5, 6, 7 or 8-fluoronaphthyl and the like; a mono or di(hydroxy)naphthyl group such as 1, 2, 3, 4, 5, 6, 7 or 8-hydroxynaphthyl, 2,4-dihydroxynaphthyl, the protected-hydroxy derivatives thereof and the like; a nitronaphthyl group such as 3- or 4-nitronaphthyl; a cyanonaphthyl group, for example, 1, 2, 3, 4, 5, 6, 7 or 8-cyanonaphthyl; a mono- or di(alkyl)naphthyl group such as 2, 3, 4, 5, 6, 7 or 8-methylnaphthyl, 1,2, 4-dimethylnaphthyl, 1, 2, 3, 4, 5, 6, 7 or 8-(isopropyl)naphthyl, 1, 2, 3, 4, 5, 6, 7 or 8-ethylnaphthyl, 1, 2, 3, 4, 5, 6, 7 or 8-(n-propyl)naphthyl and the like; a mono or di(alkoxy)naphthyl group, for example, 2,6-dimethoxynaphthyl, 1, 2, 3, 4, 5, 6, 7 or 8-methoxynaphthyl, 1, 2, 3, 4, 5, 6, 7 or 8-ethoxynaphthyl, 1, 2, 3, 4, 5, 6, 7 or 8-(isopropoxy)naphthyl, 1, 2, 3, 4, 5, 6, 7 or 8-(t-butoxy)naphthyl, 3-ethoxy-4-methoxynaphthyl and the like; 1, 2, 3, 4, 5, 6, 7 or 8-trifluoromethylnaphthyl; a mono- or dicarboxynaphthyl or (protected carboxy)naphthyl group such as 1, 2, 3, 4, 5, 6, 7 or 8-carboxynaphthyl or 2,4-di(-protected carboxy)naphthyl; a mono-or di(hydroxymethyl)naphthyl or (protected hydroxymethyl)naphthyl such as 1, 2, 3, 4, 5, 6, 7 or 8-(protected hydroxymethyl)naphthyl or 3,4-di(hydroxymethyl)naphthyl; a mono- or di(amino)naphthyl or (protected amino)naphthyl such as 1, 2, 3, 4, 5, 6, 7 or 8-(amino)naphthyl or 2,4-(protected amino)-naphthyl, a mono- or di(aminomethyl)naphthyl or (protected aminomethyl)naphthyl such as 2,3, or 4-(aminomethyl)naphthyl or 2,4-(protected aminomethyl)-naphthyl; or a mono- or di-(N-methylsulfonylamino) naphthyl such as 1, 2, 3, 4, 5, 6, 7 or 8-(N-methylsulfonylamino)naphthyl. Also, the term “substituted naphthyl” represents disubstituted naphthyl groups wherein the substituents are different, for example, 3-methyl-4-hydroxynaphth-1-yl, 3-chloro-4-hydroxynaphth-2-yl, 2-methoxy-4-bromonaphth-1-yl, 4-ethyl-2-hydroxynaphth-1-yl, 3-hydroxy-4-nitronaphth-2-yl, 2-hydroxy-4-chloronaphth-1-yl, 2-methoxy-7-bromonaphth-1-yl, 4-ethyl-5-hydroxynaphth-2-yl, 3-hydroxy-8-nitronaphth-2-yl, 2-hydroxy-5-chloronaphth-1-yl and the like.

The term “C₁ to C₈ alkyl” denotes such radicals as methyl, ethyl, n-propyl, isopropyl, n-butyl, iso-butyl, sec-butyl, tert-butyl, amyl, tert-amyl, hexyl, n-heptyl, 2-heptyl, 3-heptyl, 4-heptyl, 2-methyl-1 hexyl, 2-methyl-2hexyl, 2-methyl-3-hexyl, n-octyl and the like.

Examples of the above substituted alkyl groups include the 2-oxo-prop-1-yl, 3-oxo-but-1-yl, cyanomethyl, nitromethyl, chloromethyl, hydroxymethyl, tetrahydropyranyloxymethyl, trityloxymethyl, propionyloxymethyl, amino, methylamino, aminomethyl, dimethylamino, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, N-acetyl-2-aminoethyl, N-benzoyl-1-aminoethyl, N-acetyl-1-aminoethyl and the like.

The term “C₁ to C₈ substituted alkyl” denotes that the above C₁ to C₈ alkyl groups are substituted by one or more, and preferably one or two, halogen, hydroxy, protected hydroxy, oxo, protected oxo, C₃ to C₇ cycloalkyl, naphthyl, amino, protected amino, (monosubstituted)amino, protected (monosubstituted)amino, (disubstituted)amino, guanidino, protected guanidino, heterocyclic ring, substituted heterocyclic ring, imidazolyl, indolyl, pyrrolidinyl, C₁ to C₇ alkoxy, C₁ to C₇ acyl, C₁ to C₇ acyloxy, nitro, carboxy, protected carboxy, carbamoyl, carboxamide, protected carboxamide, N—(C₁ to C₆ alkyl)carboxamide, protected N—(C₁ to C₆ alkyl)carboxamide, N,N-di(C₁ to C₆ alkyl)carboxamide, cyano, methylsulfonylamino, thiol, C₁ to C₄ alkylthio or C₁ to C₄ alkylsulfonyl groups. The substituted alkyl groups may be substituted once or more, and preferably once or twice, with the same or with different substituents.

The term “C₃ to C₈ cycloalkyl” denotes saturated or unsatured cyclic alkyl residues such as cyclopropyl, cycloprop-[1,2]en-yl, cycloprop-[1,3]en-yl, cyclobutyl, cyclobut-[1,2]en-yl, cyclobut-[2,3]en-yl, cyclopentyl, cyclopent-[2,3]en-yl, cyclopent-[2,3-4,5]dien-yl, cyclopent-[3,4]en-yl, cyclohexyl, cycloheptyl, cyclooctyl or unsaturated derivatives of cyclohexyl, cycloheptyl or cyclooctyl.

The term “C₃ to C₈ substituted cycloalkyl” denotes denotes optionally substituted three-membered to eight-membered rings that have 1 to 4 heteroatoms, such as oxygen, sulfur and/or nitrogen, in particular nitrogen, either alone or in conjunction with sulfur or oxygen ring atoms. These five-membered to eight-membered rings may be saturated or partially unsaturated, with fully saturated rings being preferred. Preferred heterocyclic rings include tetrahydropyrimidino, perhydropyridino, dioxano, morpholino, piperidinyl, piperazinyl, 2-amino-imidazoyl, tetrahydrofurano, pyrrolo, tetrahydrothiophen-yl, hexylmethyleneimino and heptylmethyleneimino.

The term “C₇ to C₁₂ phenylalkyl” denotes a C₁ to C₆ alkyl group substituted at any position by a phenyl, substituted phenyl, heteroaryl or substituted heteroaryl. Examples of such a group include benzyl, 2-phenylethyl, 3-phenyl(n-propyl), 4-phenylhexyl, 3-phenyl(n-amyl), 3-phenyl(sec-butyl) and the like. Preferred C₇ to C₁₂ phenylalkyl groups are the benzyl and the phenylethyl groups.

The term “C₇ to C₁₂ substituted phenylalkyl” denotes a C₇ to C₁₂ phenylalkyl group substituted on the C₁ to C₆ alkyl portion with one or more, and preferably one or two, groups chosen from halogen, hydroxy, protected hydroxy, oxo, protected oxo, amino, protected amino, (monosubstituted)amino, protected (monosubstituted)amino, (disubstituted)amino, guanidino, protected guanidino, heterocyclic ring, substituted heterocyclic ring, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, nitro, carboxy, protected carboxy, carbamoyl, carboxamide, protected carboxamide, N—(C₁ to C₆ alkyl)carboxamide, protected N—(C₁ to C₆ alkyl)carboxamide, N, N—(C₁ to C₆ dialkyl)carboxamide, cyano, N—(C₁ to C₆ alkylsulfonyl)amino, thiol, C₁ to C₄ alkylthio, C₁ to C₄ alkylsulfonyl groups; and/or the phenyl group may be substituted with one or more, and preferably one or two, substituents chosen from halogen, hydroxy, protected hydroxy, cyano, nitro, C₁ to C₆ alkyl, C₁ to C₆ substituted alkyl, C₁ to C₇ alkoxy, C₁ to C₇ substituted alkoxy, C₁ to C₇ acyl, C₁ to C₇ substituted acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl) carboxamide, protected N—(C₁ to C₆ alkyl) carboxamide, N,N-di(C₁ to C₆ alkyl)carboxamide, trifluoromethyl, N—((C₁ to C₆ alkyl)sulfonyl)amino, N-(phenylsulfonyl)amino, cyclic C₂ to C₇ alkylene or a phenyl group, substituted or unsubstituted, for a resulting biphenyl group. The substituted alkyl or phenyl groups may be substituted with one or more, and preferably one or two, substituents which can be the same or different.

Examples of the term “C₇ to C₁₂ substituted phenylalkyl” include groups such as 2-phenyl-1-chloroethyl, 2-(4-methoxyphenyl)ethyl, 4-(2,6-dihydroxy phenyl)ₙ-hexyl, 2-(5-cyano-3-methoxyphenyl)n-pentyl, 3-(2,6-dimethylphenyl)n-propyl, 4-chloro-3-aminobenzyl, 6-(4-methoxyphenyl)-3-carboxy(n-hexyl), 5-(4-aminomethylphenyl)-3-(aminomethyl)n-pentyl, 5-phenyl-3-oxo-n-pent-1-yl and the like.

As outlined above R₃ and R₄ may be taken together with nitrogen to form a heterocycle or substituted heterocycle of the kind that are examplified by aziridine, azetidine, pyrrolidine, 3-methylpyrrolidine, 3-aminopyrrolidine, 3-hydroxypyrrolidine, pyrazolidine, imidazolidine, piperidine, 2-methylpiperidine, piperazine, morpholine, azepine, tetrahydroisoquinoline

The term “heterocycle” or “heterocyclic ring” denotes optionally substituted five-membered to eight-membered rings that have 1 to 4 heteroatoms, such as oxygen, sulfur and/or nitrogen, in particular nitrogen, either alone or in conjunction with sulfur or oxygen ring atoms. These five-membered to eight-membered rings may be saturated, fully unsaturated or partially unsaturated, with fully saturated rings being preferred. Preferred heterocyclic rings include morpholino, piperidinyl, piperazinyl, 2-amino-imidazoyl, tetrahydrofurano, pyrrolo, tetrahydrothiophen-yl, hexylmethyleneimino and heptylmethyleneimino.

The term “substituted heterocycle” or “substituted heterocyclic ring” means the above-described heterocyclic ring is substituted with, for example, one or more, and preferably one or two, substituents which are the same or different which substituents can be halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N—((C₁ to C₁₂ alkyl)sulfonyl)amino, N-(phenylsulfonyl)amino, heterocycle or substituted heterocycle groups.

The term “C₁ to C₈ alkoxy” as used herein denotes groups such as methoxy, ethoxy, n-propoxy, isopropoxy, n-butoxy, t-butoxy and like groups. A preferred alkoxy is methoxy. The term “C₁ to C₈ substituted alkoxy” means the alkyl portion of the alkoxy can be substituted in the same manner as described above in relation to C₁ to C₈ substituted alkyl.

The term “C₁ to C₈ aminoacyl” encompasses groups such as formyl, acetyl, propionyl, butyryl, pentanoyl, pivaloyl, hexanoyl, heptanoyl, octanoyl, benzoyl and the like.

The term “C₁ to C₈ substituted aminoacyl” denotes the acyl group substituted by one or more, and preferably one or two, halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C₁ to C₁₂ alkoxy, C₁ to C₁₂ acyl, C₁ to C₁₂ acyloxy, nitro, C₁ to C₁₂ alkyl ester, carboxy, protected carboxy, carbamoyl, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, cyano, methylsulfonylamino, thiol, C₁ to C₁₀ alkylthio or C₁ to C₁₀ alkylsulfonyl groups. The substituted acyl groups may be substituted once or more, and preferably once or twice, with the same or with different substituents.

Examples of C₁ to C₈ substituted acyl groups include 4-phenylbutyroyl, 3-phenylbutyroyl, 3-phenylpropanoyl, 2-cyclohexanylacetyl, cyclohexanecarbonyl, 2-furanoyl and 3-dimethylaminobenzoyl.

This invention provides a pharmaceutical composition comprising an effective amount of a compound according to the invention. Such compounds can be administered by various routes, for example oral, subcutaneous, intramuscular, intravenous or intracerebral. The preferred route of administration would be oral at daily doses of the compound for adult human treatment of about 0.01-5000 mg, preferably 1-1500 mg per day. The appropriate dose may be administered in a single dose or as divided doses presented at appropriate intervals for example as two, three four or more subdoses per day.

For preparing pharmaceutical compositions containing compounds of the invention, inert, pharmaceutically acceptable carriers are used. The pharmaceutical carrier can be either solid or liquid. Solid form preparations include, for example, powders, tablets, dispersible granules, capsules, cachets, and suppositories.

A solid carrier can be one or more substances which can also act as diluents, flavoring agents, solubilizers, lubricants, suspending agents, binders, or tablet disintegrating agents; it can also be an encapsulating material.

In powders, the carrier is generally a finely divided solid which is in a mixture with the finely divided active component. In tablets, the active compound is mixed with the carrier having the necessary binding properties in suitable proportions and compacted in the shape and size desired.

For preparing pharmaceutical composition in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter and the like.

The pharmaceutical compositions can include the formulation of the active compound with encapsulating material as a carrier providing a capsule in which the active component (with or without other carriers) is surrounded by a carrier, which is thus in association with it. In a similar manner, cachets are also included. Tablets, powders, cachets, and capsules can be used as solid dosage forms suitable for oral administration.

Liquid pharmaceutical compositions include, for example, solutions suitable for oral or parenteral administration, or suspensions, and emulsions suitable for oral administration. Sterile water solutions of the active component or sterile solutions of the active component in solvents comprising water, ethanol, or propylene glycol are examples of liquid compositions suitable for parenteral administration.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

In one embodiment of the present invention a compound is claimed according to formula (1) above, or pharmaceutical acceptable salts or solvates thereof, wherein R₁ is H, R₂ is substituted phenyl, C₅ to C₆ heteroaryl, or C₅ to C₆ substituted heteroaryl, R₃ is H, R₄ in formula (1) is a structure according to formula (4) shown below, R₅ is H or a halogen, R₆ is H and R₇ is H. 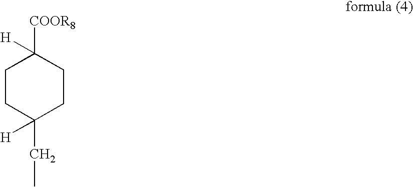
wherein the COOR₈ and the methylene substituents can adopt all possible diastereomeric configurations.
The term “all possible diastereomeric configurations” is meant to encompass all combinations of equatorial and axial configurations of substituents.
In a preferred embodiment of the invention a compound is claimed, or pharmaceutical acceptable salts or solvates thereof, wherein R₁ is H, R₂ is substituted phenyl, R₄ is H, R₃ in formula (5) is a structure according to formula (6) shown below, R₅ is H, R₆ is H and R₇ is H.
R₈ is H, methyl or ethyl. 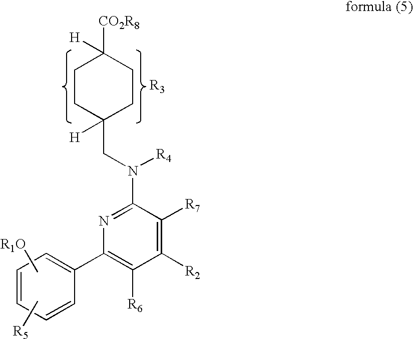
wherein:
R₁ is preferred as H
R₂ is preferred as substituted phenyl, C₅ to C₆ heteroaryl, or C₅ to C₆ substituted heteroaryl
R₄ is H and R₃ is preferred as of the formula (6) 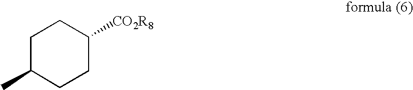
wherein the COOR₈ and the methylene substituents are in double axial (a,a) positions.
R₈ is H, methyl or ethyl.

A particularly preferred compound which may act as agonist of NR1H4 is shown in formula (7) below. The inventors have been able to demonstrate that the compound according to formula (7) has a low effective concentration at FXR with an EC₅₀ of 0,04 μM wherein the EC₅₀ reflects the half-maximal effective concentration, and which is higher than the EC₅₀ of 0,015 μM for the published FXR agonist GW4064 (B. Goodwin et al., Molecular Cell 6, 517-526, 2000)

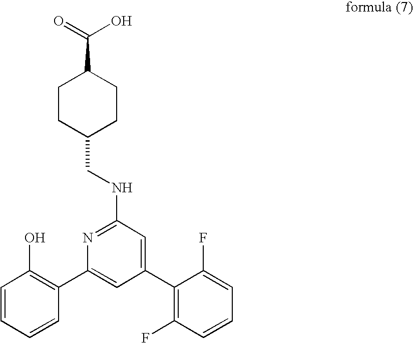

The inventors have also found the compounds according to formulae (8 to 14) (shown below) to be active as agonists of the NR1H4 human nuclear receptor (see figures for details).

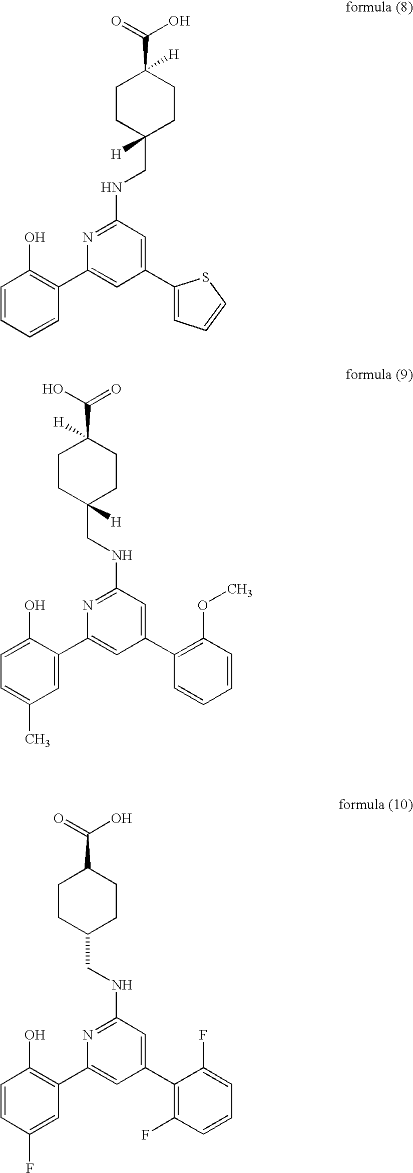

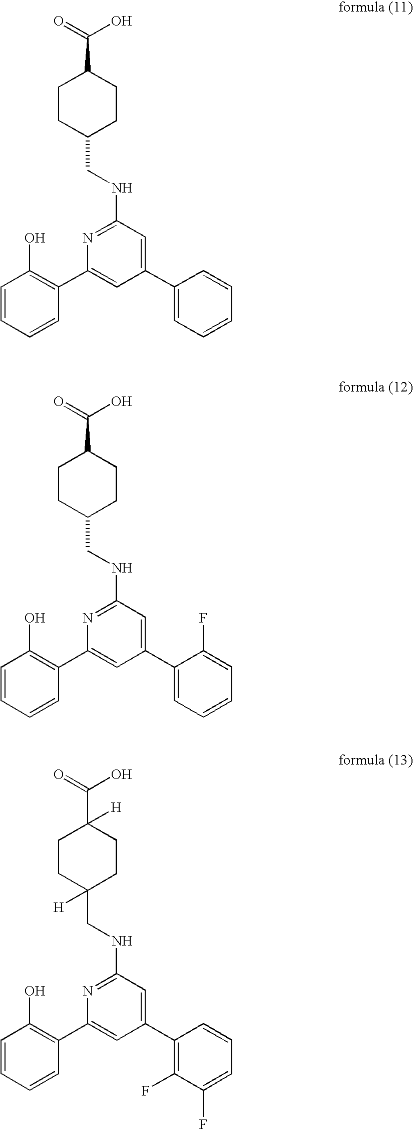

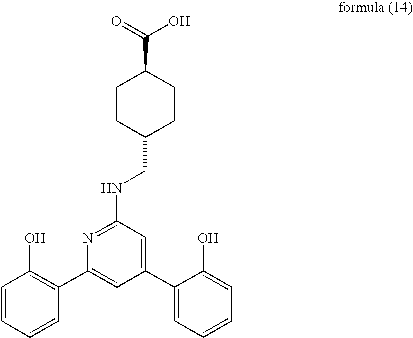

In particular the invention relates to a compound as described above wherein said compounds is capable of binding the NR1H4 receptor protein or a portion thereof according to SEQ ID NO. 1 (FIG. 3 A to D) or a mammalian homologue thereof. The claimed compound can bind to the NR1H4 receptor protein or a portion thereof in a mixture comprising 10-200 ng of NR1H4 receptor protein or a portion thereof, preferably the ligand binding domain, 20 mM Tris /HCl at pH 7.9; 60 mM KCl; 5 mM MgCl2; 160 ng/μl BSA in a total volume of preferably about 25 μl.

A mammalian receptor protein homologue of the protein according to SEQ ID NO. 1 as used herein is a protein that performs substantially the same task as NR1H4 does in humans and shares at least 40% sequence identity at the amino acid level, preferably 50% sequence identity at the amino acid level more preferably 65% sequence identity at the amino acid level, even more preferably 75% sequence identity at the amino acid level and most preferably over 85% sequence identity at the amino acid level.

The invention in particular concerns a method for prevention or treatment of a NR1H4 receptor protein or NR1H4 receptor protein homologue mediated disease or condition in a mammal comprising administration of a therapeutically effective amount of a compound according to the invention wherein the prevention or treatment is directly or indirectly accomplished through the binding of a compound according to the invention to the NR1H4 receptor protein or to the NR1H4 receptor protein homologue.

The term mediated herein means that the physiological pathway in which the NR1H4 receptor protein acts is either directly or indirectly involved in the disease or condition to be treated or prevented. In the case where it is indirectly involved it could be that, e.g. modulating the activity of NR1H4 by a compound according to the invention influences a parameter which has a beneficial effect on a disease or a condition. One such example is that modulation of NR1H4 activity leads to decreased levels of serum cholesterol or certain lipoproteins which in turn have a beneficial effect on the prevention and treatment of artherosclerosis. Herein a condition is a physiological or phenotypic state which is desirably altered. Another example would be the treatment of cholestatic conditions in which bile flow from the liver to the gut is impaired which results in a tailback of toxic metabolites to the liver. Cholestasis can be a primary condition where bile flow is directly impaired or a secondary condition where a primary impairment in liver function such as liver cirrhosis results in a secondary cholestasis. Agonists that activate NR1H4 resulting in increased bile acid export from the hpeatocyte into the liver canaliculi and subsequent increased bile flow might be used for the treatment of these different types of cholestasis.

In a preferred embodiment of the invention the method for prevention or treatment of a NR1H4 receptor protein mediated disease or condition is applied to a human. This may be male or female.

Pharmaceutical compositions generally are administered in an amount effective for treatment or prophylaxis of a specific condition or conditions. Initial dosing in human is accompanied by clinical monitoring of symptoms, such symptoms for the selected condition. In general, the compositions are administered in an amount of active agent of at least about 100 μg/kg body weight. In most cases they will be administered in one or more doses in an amount not in excess of about 20 mg/kg body weight per day. Preferably, in most cases, doses is from about 100 μg/kg to about 5 mg/kg body weight, daily.

For administration particularly to mammals, and particularly humans, it is expected that the daily dosage level of active agent will be 0,1 mg/kg to 10 mg/kg and typically around 1 mg/kg.

By “therapeutically effective amount” is meant a symptom-alleviating or symptom-reducing amount, a cholesterol-reducing amount, an amount that overcomes cholestatic conditions, a protein and/or carbohydrate digestion-blocking amount and/or a de novo cholesterol biosynthesis-blocking amount of a compound according to the invention.

FXR is proposed to be a bile acid sensor. As a result, it modulates both, the synthetic output of bile acids from the liver and their recycling in the intestine, by regulating bile acid binding proteins. In one embodiment of the invention the invention concerns a method for regulating the bile transport system in a mammal, in a preferred embodiment a human, which comprises activating the NR1H4 receptor with a therapeutically effective amount of a compound according to the invention.

Likewise the invention concerns a method of treating in mammal a disease which is affected by cholesterol, triglyceride, bile acid levels or bile flow comprising administering to a mammal in need of such treatment a therapeutically effective amount of a compound according to the invention.

Accordingly, the compounds according to the invention may also be used as a method of prevention or treatment of mammalian atherosclerosis, gallstone disease (cholelithiasis), primary and secondary forms of cholestasis, lipid disorders, obesity or cardiovascular disorders such as coronary heart disease or stroke.

The invention further concerns a method of blocking in a mammal the cholesterol absorption in the intestine of a mammal in need of such blocking comprising administering to a mammal in need of such treatment a therapeutically effective amount of a compound according to the invention. The invention may also be used to treat obesity in humans.

The Farnesoid X Receptor alpha is a prototypical type 2 nuclear receptor which activates genes upon binding to the promoter region of target genes in a heterodimeric fashion with Retinoid X Receptor. The relevant physiological ligands of NR1H4 are bile acids. The present compounds according to the invention have been demonstrated to have a high binding efficacy as measured as IC50 in the range 400 nM to 1000 nM as well as agonistic and/or antagonistic properties. Consequently they may be applied to regulate genes that participate in bile acid homeostasis as well as other downstream regulated genes. Examples of physiological functions in which such genes are involved are but are not limited to lipid absorption, cholesterol biosynthesis, cholesterol transport or binding, bile acid synthesis, bile acid transport or binding, proteolysis, amino acid metabolism, glucose biosynthesis, protein translation, electron transport, and hepatic fatty acid metabolism. FXR often functions in vivo as a heterodimer with the Retinoid X Receptor. Published FXR agonists such as the Glaxo SmithKline compound “GW 4064” and published FXR antagonists such as guggulsterone [4, 17(20) -pregnadiene-3, 16-dione] are known to influence the regulation of various liver genes. Genes found to be regulated by GW 4064 can be found in FIG. 6. Thus, the invention also concerns a method of modulating a gene whose expression is regulated by the NR1H4 receptor in a mammal comprising administration of a therapeutically effective amount of a compound according to the invention to said mammal.

It is known that the orphan receptor FXR can bind the response element of the shp gene as a heterodimer with RXR (9-cis retinoic acid receptor) and the SHP-protein, in turn, prevents efficient transcription from the cyp7a1 promoter (Lu et al., Mol Cell, 6(3):505-17; Goodwin et al. Mol Cell, 6(3), 717-26, 2000). Another gene that is repressed via SHP upon FXR activation is the Sodium/Bile Acid Cotransporter gene ntcp, a membrane transport protein which is required for the import of conjugated bile acids into the hepatocyte (Denson et al., Gastroenterology;121(1):218-20, 2001). The gene for the Bile Salt Export Pump, a membrane transporter responsible for the secretion of bile acids into the gall is directly activated by FXR (Ananthanarayanan et al., J Biol Chem, 3;276(31):28857-28865, 2001). Consequently, the invention likewise concerns a method for lowering the expression of cholesterol 7-alpha-hydroxylase and NTCP and increasing expression of BSEP and/or MDR2 (=multidrug resistance protein 2) in parallel by use of the compounds according to the invention. This is believed to be the ideal profile of an anti-cholestatic compound (Kullack-Ublick, et al., J Hepatol (2000) 32 Suppl 1:3-18). In one embodiment the invention concerns a method for enhancing the expression of the Intestinal Bile Acid Binding Protein (I-BABP) (Grober et al., J Biol Chem, 15;274(42):29749-54, (1999) and/or the activity of the canicular bile salt excretion pump.

The compounds according to the invention may be used as medicaments, in particular for the manufacture of a medicament for the prevention or treatment of a NR1H4 receptor protein or NR1H4 receptor protein homologue mediated disease or condition in a mammal wherein the prevention or treatment is directly or indirectly accomplished through the binding of the compound according to the invention to the NR1H4 receptor protein or NR1H4 receptor protein homologue. These pharmaceutical compositions contain 0,1% to 99,5% of the compound according to the invention, more particularly 0,5% to 90% of the compound according to the invention in combination with a pharmaceutically acceptable carrier.

The invention concerns also the use of a compound according to the invention for the manufacture of a medicament for the prevention or treatment of a NR1H4 receptor protein mediated disease or condition wherein the mammal described above is a human. The medicament may be used for regulating the bile transport system in a mammal preferentially a human by activating the NR1H4 receptor, for regulating levels of cholesterol, triglyceride, bile acids and bile flow in mammals, preferentially humans. The medicament may be used for the treatment of atherosclerosis, gallstone disease (cholelithiasis), cholestasis, lipid disorders, obesity or a cardiovascular disorder.

The further concerns the use of a compound according to the invention for the manufacture of a medicament capable for blocking in a mammal, preferentially a human the cholesterol absorption in the intestine. Further the claimed compound may be used for the manufacture of a medicament for treating obesity in humans and for modulating a gene whose expression is regulated by the NR1H4 receptor (see details above and figures). The invention further concerns the use of a compound according to the invention for the manufacture of anticancer medicaments. The anticancer effects of such medicaments could be excerted by selective inhibition of cell proliferation and induction of apoptosis of tumor cells in a way similar to described activities for certain bisphosphonates (Alberts D S, et al., Clin Cancer Res 2001 May; 7(5):1246-50.

To describe the invention in its essence:

The object of the invention is solved by a compound including resolved diastereoisomers and enantiomers, and tautomers of the formula (1), or pharmaceutical acceptable salts or solvates thereof, 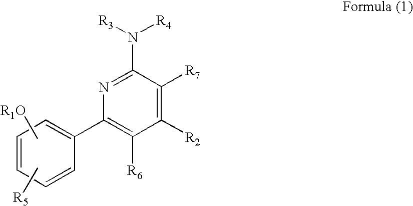
wherein:
R₁ in formula (1) is H, C₁ to C₇ acyl or C₁ to C₇ substituted acyl,
R₂ is phenyl, substituted phenyl, C₅ to C₆ heteroaryl, C₅ to C₆ substituted heteroaryl, naphthyl or substituted naphthyl,
R₃ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl, C₇ to C₁₂ substituted phenylalkyl, or phenyl,
R₄ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl or C₇ to C₁₂ substituted phenylalkyl,
R₃ and R₄ may be taken together with nitrogen to form a heterocycle or substituted heterocycle, or a heteroaryl or substituted heteroaryl ring,
R₅ is H, C₁ to C₈ alkyl, halogen, hydroxy, alkoxy, in particular C₁ to C₈ alkoxy, carboxy, ester, amide or C₁ to C₈ aminoacyl,
R₆ is H, C₁ to C₈ alkyl, C₁ to C₈ substituted alkyl and
R₇ is H, F, Cl, methyl, or trifluoromethyl.

In a preferred embodiment of the compound according to the present invention

R₁ is H,

R₂ is substituted phenyl, C₅ to C₆ heteroaryl, substituted C₅ to C₆ heteroaryl,

R₃ is H,

R₄ in formula (1) is a structure according to formula (2),
—CH₂—R₄₋₁—CO₂R₈ formula (2)
,wherein:
R₄₋₁ is C₁ to C₈ alkyl, C₁ to C₈ substituted alkyl, C₃ to C₈ cycloalkyl, C₃ to C₈ substituted cycloalkyl, C₅ to C₆ aryl, or C₅ to C₆ substituted aryl, C₅ to C₆ heteroaryl, or C₅ to C₆ substituted heteroaryl,
and methylene and the COOR₈ substituents take the [1,4]-positions in case R₄₋₁ is cyclohexyl, substituted cyclohexyl, C₆ aryl, C₆ substituted aryl, C₆ heteroaryl, or C₆ substituted heteroaryl, or methylene and the COOR₈-substituents take the [1,3]-positions in case R₄₋₁ is cyclopentyl, substituted cyclopentyl, or substituted C₅ heteroaryl,
methylene and the COOR₈ substituents can have all possible diastereomeric configurations,
R₅ is H or a halogen, hydroxy, alkoxy or C₁ to C₈ alkyl,
R₆ is H and
R₇ is H.
R₈ is H, methyl or ethyl.

In one embodiment of the present invention

R₃ is H,

R₄ in formula (1) is a structure according to formula (3),
—R₄₋₁—CO₂R₈ formula (3)
,wherein:
R₄₋₁ is C₁ to C₈ alkyl, C₁ to C₈ substituted alkyl, C₃ to C₈ cycloalkyl, C₃ to C₈ substituted cycloalkyl, C₅ to C₆ aryl, or C₅ to C₆ substituted aryl, C₅ to C₆ heteroaryl, or C₅ to C₆ substituted heteroaryl,
   and methylene and the COOR₈ substituents take the [1,4]-positions in case R₄₋₁ is cyclohexyl, substituted cyclohexyl, C₆ aryl, C₆ substituted aryl, C₆ heteroaryl, or C₆ substituted heteroaryl, or methylene and the COOR₈-substituents take the [1,3]-positions in case R₄₋₁ is cyclopentyl, substituted cyclopentyl, or substituted C₅ heteroaryl,
   methylene and the COOR₈ substituents can have all possible diastereomeric configurations.

In a preferred embodiment of the compound according to the present invention

R₁ is H,

R₂ is substituted phenyl,

R₃ is H,

R₄ in formula (1) is a structure according to formula (4), 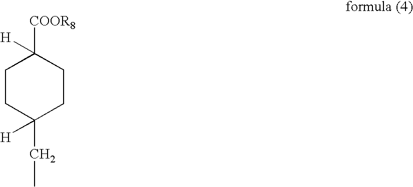
methylene and the COOR₈ substituents can have all possible diastereomeric configurations,
R₅ is H or a halogen, hydroxy, alkoxy or C₁ to C₈ alkyl,
R₆ is H and
R₇ is H.
R₈ is H, methyl or ethyl.

In one embodiment of the present invention, the compound can be represented by 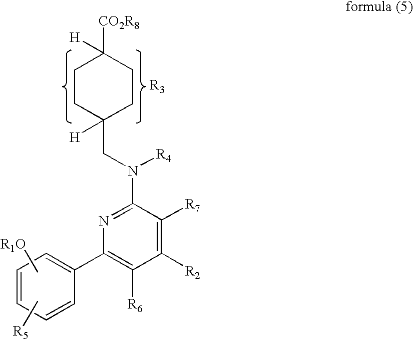
wherein:
R₁ is H,
R₂ is substituted phenyl, C₅ to C₆ heteroaryl, or C₅ to C₆ substituted heteroaryl,
R₄ is H and R₃ is a structure according to formula (6) 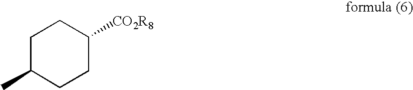
wherein the COOR₈ and the methylene substituents are in double axial (a,a) positions,
R₅ is H or a halogen, hydroxy, alkoxy or C₁ to C₈ alkyl,
R₆ is H
R₇ is H
R₈ is H, methyl or ethyl.

In a preferred embodiment of the compound according to the present invention it can be represented by

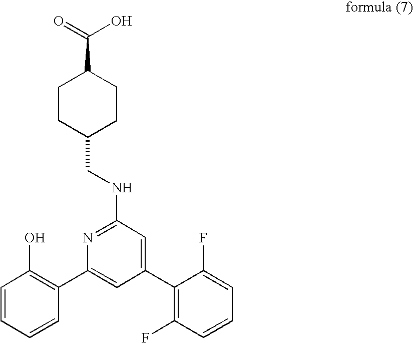

In a preferred embodiment of the compound according to the present invention it can be represented by

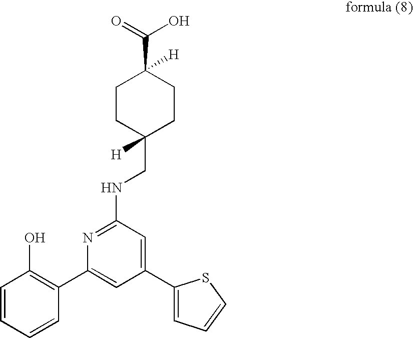

In a preferred embodiment of the compound according to the present invention it can be represented by

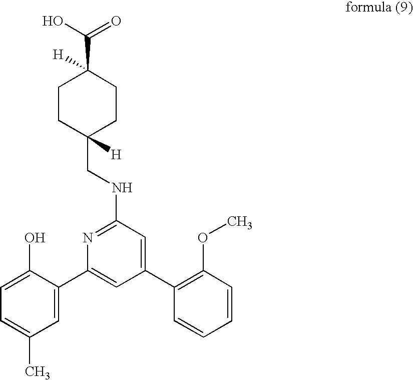

In a preferred embodiment of the compound according to the present invention it can be represented by

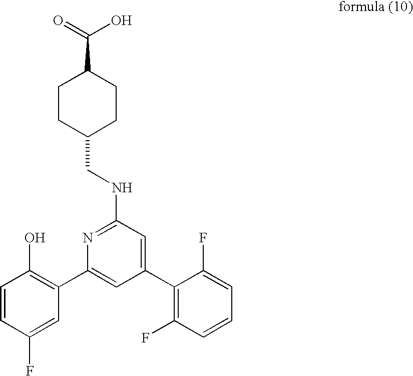

In a preferred embodiment of the compound according to the present invention it can be represented by

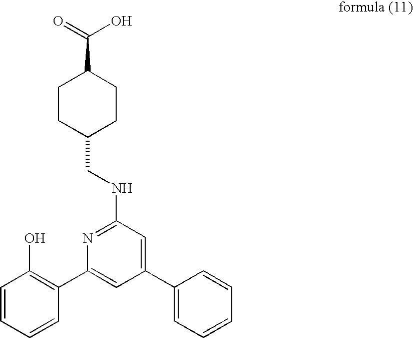

In a preferred embodiment of the compound according to the present invention it can be represented by

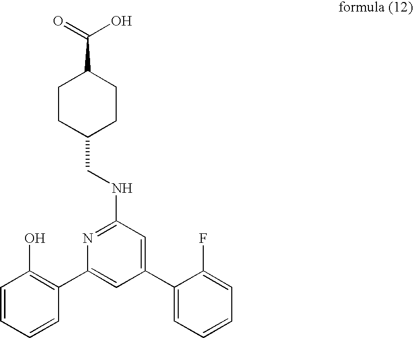

In a preferred embodiment of the compound according to the present invention it can be represented by

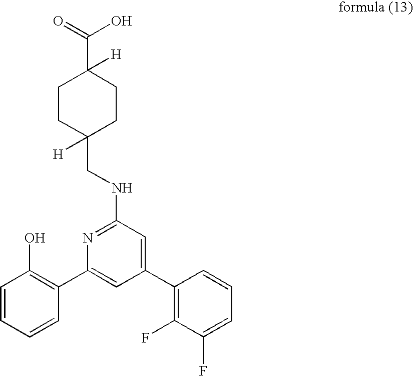

In a preferred embodiment of the compound according to the present invention it can be represented by

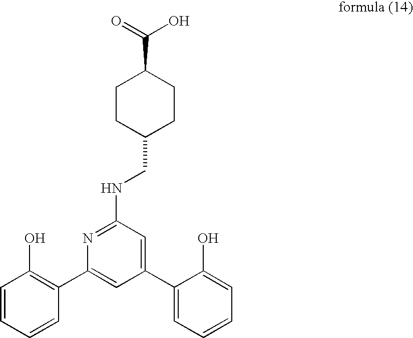

The object of the invention is also solved by a compound as defined above for use as a medicament.

The object of the present invention is also solved by a compound according the present invention wherein said compound is capable of binding the human NR1H4 receptor protein or a portion thereof or a mammalian homologue thereof according to SEQ ID NO. 1.

The object of the present invention is also solved by a compound according the present invention wherein it is used for the manufacture of a medicament for the treatment of a NR1H4 receptor mediated or treatable disease or condition in a mammal comprising administration of a therapeutically effective amount of a compound according to the present invention.

The object of the present invention is also solved by a compound according the present invention wherein it is used for the manufacture of a medicament for regulating bile flow or the bile acid transport system in a mammal by activating or repressing the NR1H4 receptor with a therapeutically effective amount of a compound according to the present invention.

The object of the present invention is also solved by a compound according the present invention wherein it is used for the manufacture of a medicament for regulating blood levels of cholesterol, lipoproteins, phospholipids, triglycerides, or bile acids and/or bile flow or bile levels of cholesterol, phospholipids or bile acids in a mammal in need of such treatment with a therapeutically effective amount of a compound according to the present invention.

The object of the present invention is also solved by a compound according the present invention wherein it is used for the manufacture of a medicament for treating cholestatic conditions such as primary biliary cirrhosis (PBC), progressive familiary cholestasis (PFIC), estrogen or drug induced cholestasis, any form of extrahepatic cholestasis, or secondary forms of cholestasis, atherosclerosis, gallstone disease (cholelithiasis), lipid disorders, obesity or a cardiovascular or metabolic disorder in a mammal in need of such treatment with a therapeutically effective amount of a compound according to the present invention.

The object of the present invention is also solved by a compound according the present invention wherein it is used for the manufacture of a medicament for treating in a mammal malign proliferative diseases such as cancer which can be treated or cured by inducing apoptosis in the affected cells or tissues comprising administering to a mammal in need of such treatment a therapeutically effective amount of a compound according to the present invention.

The object of the present invention is also solved by a compound according the present invention wherein it is used for the manufacture of a medicament for treating in a mammal conditions of drug resistance that arise during drug treatment of disorders such as cancer or infectious diseases, or during continous administration of contraceptive drugs comprising administering to a mammal in need of such treatment a therapeutically effective amount of a compound according to the present invention.

The object of the present invention is also solved by a compound according the present invention wherein it is used for the manufacture of a medicament capable of blocking in a mammal cholesterol absorption or capable of reducing the bile acid reabsorption in the intestine in a mammal in need of such treatment comprising administration of a therapeutically effective amount of a compound according to the present invention.

The object of the present invention is also solved by a compound according the present invention wherein it is used for the manufacture of a medicament for regulating the expression of NR1H4 responsive genes such as cholesterol-7-alpha hydroxylase (cyp7a1), sterol-12-alpha hydroxylase (cyp8b1), small heterodimer partner (shp), phospholipid transfer protein (pltp), bile salt export pump (bsep), sodium-taurocholate co-transporter (ntcp), organic anion transport proteins 1 and 2 (oatp1 and -2), canalicular multidrug resistance protein 2 (mdr2) or other genes that are members of the cytochrom P450 family or members of the ABC-transporter family or members of the MDR class III multidrug resistance proteins or members of the MRP multidrug resistance protein family or members of the nuclear receptor gene family through activating or repressing the NR1H4 receptor in a mammal in need of such treatment by a therapeutically effective amount of a compound according to the present invention.

The object of the present invention is also solved by a compound according the present invention wherein it is used for the manufacture of a medicament for modulating the expression of the intestinal bile acid binding protein (IBABP) in intestinal mucosa cells and/or cholangiocytes by the NR1H4 receptor in a mammal in need of such treatment by a therapeutically effective amount of a compound according to the present invention.

In a preferred embodiment of the use according to the present invention the mammal is a human.

The object of the present invention is furthermore solved by a method for prevention or treatment of a disease or condition which is mediated or can be adressed by the NR1H4 receptor in a mammal comprising administration of a therapeutically effective amount of a compound according to the present invention.

The object of the present invention is furthermore solved by a method for regulating bile flow or the bile acid transport system in a mammal which comprises activating or repressing the NR1H4 receptor with a therapeutically effective amount of a compound according to the present invention.

The object of the present invention is furthermore solved by a method of treating in a mammal a disease or condition which is affected by impaired blood levels of cholesterol, lipoproteins, phospholipids, triglycerides, or bile acids and/or impaired bile flow or impaired bile levels of cholesterol, phospholipids or bile acids comprising administering to a mammal in need of such treatment a therapeutically effective amount of a compound according to the present invention.

The object of the present invention is furthermore solved by a method of treating in a mammal cholestatic conditions such as primary biliary cirrhosis (PBC), progressive familiary cholestasis (PFIC), estrogen or drug induced cholestasis, any form of extrahepatic cholestasis, or secondary forms of cholestasis, atherosclerosis, gallstone disease, lipid disorders, obesity or a cardiovascular or metabolic disorder comprising administering to a mammal in need of such treatment a therapeutically effective amount of a compound according to the present invention.

The object of the present invention is furthermore solved by a method for treating in a mammal malign proliferative diseases such as cancer which can be treated by inducing apoptosis in the affected cells or tissues comprising administering to a mammal in need of such treatment a therapeutically effective amount of a compound according to the present invention.

The object of the present invention is furthermore solved by a method for treating in a mammal conditions of drug resistance that arise during drug treatment of disorders such as cancer or infectious diseases, or during continous administration of contraceptive drugs comprising administering to a mammal in need of such treatment a therapeutically effective amount of a compound according to the present invention.

The object of the present invention is furthermore solved by a method of blocking in a mammal the cholesterol absorption or bile acid re-absorption in the intestine of a mammal in need of such blocking comprising administering to a mammal in need of such treatment a therapeutically effective amount of a compound according to the present invention.

The object of the present invention is furthermore solved by a method according to the present invention for regulating the expression of NR1H4 responsive genes such as cholesterol-7-alpha hydroxylase (cyp7a1), sterol-12-alpha hydroxylase (cyp8b1), small heterodimer partner (shp), phospholipid transfer protein (pltp), bile salt export pump (bsep), sodium-taurocholate co-transporter (ntcp), organic anion transport proteins 1 and 2 (oatp1 and -2), canalicular multidrug resistance protein 2 (mdr2) or other genes that are members of the cytochrom P450 family or members of the ABC-transporter family or members of the MDR class III multidrug resistance proteins or members of the MRP multidrug resistance protein family or members of the nuclear receptor gene family by the NR1H4 receptor in a mammal comprising administering a therapeutically effective amount of a compound according to the present invention.

The object of the present invention is furthermore solved by a method for modulating the expression of the intestinal bile acid binding protein (IBABP) in intestinal mucosa cells and/or cholangiocytes by the NR1H4 receptor in a mammal comprising administering a therapeutically effective amount of a compound according to the present invention.

In a preferred embodiment of the method according to the present invention, the mammal is a human.

The examples that follow are not intended to limit the scope of the present invention but are merely intended to illustrate it.

### EXAMPLES

### Example 1

In vitro screening for compounds which influence FXR binding to coactivators.

For screening purposes a fragment of the open reading frame of human FXR alpha (NR1H4—(Acc. No:AF384555)) encoding aminoacids 187-472 was amplified by standard RT PCR procedures (see figures; SEQ ID NO. 1 and 2). Starting material was total RNA derived from human liver. The resulting cDNA obtained after reverse transcription was subsequently cloned using the Gateway™ recombination technology (Invitrogen, USA) into the expression plasmid pDest15 (Invitrogen, USA). This construct was used to express a recombinant GST-FXR fusion protein in E. coli (BL21 strain). A pDEST 17 derivative clone harboring an additional sequence encoding amino acids 548-878 of human TIF2 (Acc. No: XM_{—}011633 RefSeq) was constructed using Gateway™ recombination technology (Invitrogen, USA) in order to obtain a construct which was used to express recombinant His-tagged TIF2 fragment could be expressed in E. coli. For E. coli expression of both constructs, plasmid DNA was transformed into chemically competent E. coli BL21 (Invitrogen, USA) and cells were grown to an OD600 of 0.4-0.7 before expression was induced by addition of 0,5 mM IPTG according instructions of the manufacturer (Invitrogen). After induction for 8 hours at 30° C. cells were harvested by centrifugation for 10 minutes at 5000×g. Fusion proteins were affinity purified using Glutathion sepharose (Pharmacia) or Ni-NTA Agarose (QIAGEN) according to the instructions of the respective manufacturer. Recombinant proteins were dialyzed against 20 mM Tris/HCL pH 7.9; 60 mM KCl; 5 mM MgCl₂; 1 mM DTT, 0,2 mM PMSF; 10% glycerol. The TIF2 fragment was subsequently biotinylated by addition of 40-120 μl of a Biotinamidocaproate N-Hydroxysuccinimide-ester (Sigma) solution (20 mg/ml in DMSO). Overhead rotating samples were incubated for 2 hours at room temperature. Unincorporated label was then separated using G25 Gel filtration chromatography (Pharmacia Biotech, Sweden). Protein containing fractions from the column were pooled and tested for activity in the assay as described below.

For screening of compound libraries as provided for by the methods shown below in the examples for substances which influence the FXR/Tif 2 interaction, the Perkin Elmer LANCE technology was applied. This method relies on the binding dependent energy transfer from a donor to an acceptor fluorophore attached to the binding partners of interest. For ease of handling and reduction of background from compound fluorescence LANCE technology makes use of generic fluorophore labels and time resoved detection (for detailed description see Hemmilä I, Blomberg K and Hurskainen P, Time-resolved resonance energy transfer (TR-FRET) principle in LANCE, Abstract of Papers Presented at the 3 rd Annual Conference of the Society for Biomolecular Screening, Sep., California (1997).

For screening, 20-200 ng of biotinylated Tif 2 fragment and 10-200 ng of GST-FXR fragment were combined with 0.5-2 nM LANCE Eu-(W1024) labelled anti-GST antibody (Perkin Elmer) and 0,5-2 μg of Highly fluorescent APC-labelled streptavidin (Perkin Elmer) in the presence of 50 μM of individual compounds to be screened in a total volume of 25 μl of 20 mM Tris /HCl pH 7.9; 60 mM KCl; 5 mM MgCl2; 160 ng/μl BSA. DMSO content of the samples was kept below 4%. Samples were incubated for a minimum of 60 minutes in the dark at room temperature in FIA-Plates black 384well med. binding (Greiner).

The LANCE signal was detected by a Perkin Elmer VICTOR2V™ Multilabel Counter applying the detection parameters listed in FIG. 2. The results were visualized by plotting the ratio between the emitted light at 665 nm and at 615 nm. For every batch of recombinant proteins amount of proteins and labeling reagents giving the most sensitive detection of hits was determined individually by analysis of dose response curves for chenodeoxycholic acid.

### Example 2

Experimental procedure for the preparation of the compounds according to the invention.

Step 1. General Procedure for Preparation of Br-Wang Resin

1.6 g of Wang resin (1.28 mmol/g, 2.0 mmol/bag) was placed in a porous polypropylene packets (Tea-bag, 60 mm×50 mm, 65 p), sealed and transferred to a 125 ml PP bottle. A freshly prepared solution of PPh₃Br₂ (6.1 mmol, 3.0 equivalents, 0.15 M) in DCM (40 mL) was added to each packet. After shaking for 4-6 hours at room temperature, the packet was washed with DCM (5×80 ml) and diethyl ether (4×80 ml). The packet was dried overnight under vacuum to afford off-white resin.

Step 2. Reaction of Acetophenones with Br-Wang resin.

Each packet containing freshly prepared Br-Wang resin was transferred to an appropriate glass bottle, to which an Acetophenone (20 mmol, 10 equivalents, 0.2 M), anhydrous DMA (100 ml) and KOtBu (20 mmol, 10 equivalents, 0.2 M) were added sequentially. After heating at 50° C. for 24 hours, the packet was washed alternatively with DMF (3×80 ml) and MeOH (2×80 ml) followed by DCM (2×80 ml) and MeOH (3×80 ml). The packet was air-dried overnight to afford off-white to pale brown resin, depending on the Acetophenone used in the synthesis.

Step 3. Reaction of Aldehydes with Wang Resin-Bound Acetophenones.

Each packet of Acetophenone-Wang resin was transferred to a 250 mL PP bottle, to which a solution of NaOMe (40 mmol, 20 eq, 0.25 M) in 50% THF-MeOH (160 ml) and an Aldehyde (40 mmol, 20 equivalents, 0.25 M) were added sequentially. After shaking at room temperature for 3 days, the packet was washed several times with MeOH (3×80 ml) and alternatively with DMF (80 ml) and MeOH (80 ml) for 3 cycles, followed by washes of DCM (2×80 ml) and MeOH (3×80 ml). The packet was air-dried overnight to afford a resin-bound chalcone, that varied in color from yellow to dark red depending on the aldehyde used.

Step 4. In Situ Preparation of Amidines from the Reactions of BtCH₂CN and the Corresponding Amines.

The amidines required for step 5 were prepared as follows: For each well, an amine (0.5 mmol, 10 equivalents, 0.4 M), BtCH₂CN (0.5 mmol, 10 equivalents, 0.4 M) and MeOEtOH, glyme or methylethyleneglycol (1.25 ml) were added to a glass bottle and mixed until completely dissolved. After heating at 80° C. for 24 hours, the solutions of amidines were carried to step 5 without purification.

Step 5: Reaction of Amidines with the Wang Resin-Bound Chalcone.

The tea bag containing the Chalcone on Wang resin from step 3 was cut open and the resin was distributed equally into 40 wells of a microtiter. A solution of the amidine (0.5 mmol, 10 equivalents, 0.4 M) in MeOEtOH (1.25 ml) was added to the corresponding well. The plate was tightly capped, gently shaken and incubated at 75° C. for 40 hours. Each plate was washed alternatively with DMF (6×1 ml/well) and MeOH (8×1 ml/well). The plate was air-dried overnight and under vacuum for 4 hours.

Step 6. Cleavage from Linker and Extraction:

To dry microtiter plates containing the aminopyridine resins was added 0.5 ml of 20% TFA/DCM to each well. The plates were capped and placed on a shaker at room temperature for 3 hours. The plates were transferred to a GENEVAC to remove the volatile TFA/DCM solution. The plates were extracted with AcOH and the extract frozen and lyophilized to afford the title compounds. All of the final products were analyzed using an Evaporative Light Scattering Detector (ELSD) detection to determine purity.

A detailed protocol for the synthesis of various 2-aminopyridine derivatives is also given in WO 01/23887. One skilled in the art will be able to arrive at the compounds claimed herein making use of said protocol.

### Example 3

This example illustrates that a compound according to the invention (experiments shown were done with MOLSTRUCTURE LN12996 (see FIG. 4 for structural formula)) can mediate transactivation of FXR mediated transcription in a HEK293 reporter cell line.

Stable HEK293FXR reporter cell lines were generated by stably transfecting with the pTRexDest30 (Invitrogen) derivatives pTRexDest30-hFXR, pTRexDest30-hRXRα and the pGL2promoter (Promega) derivative pGL2promoter-FXRRE. The full length human FXR (accession U68233) and the full length human RXRα (accession P19793) were cloned into the pTRexDest30 applying the manufacturer protocols for the Gateway™ system (Invitrogen).

The FXR response elements were (upper case and underlined) 5′-cccaGGGTGAaTAACCTcggggctctgtccctccaatcccaGGGTGAaTAACCTcggg 3′ (SEQ ID NO. 5) was created from the human IBAB-P promoter (Grober et al 1999, JBC 274, pp. 29749-29754). A stable clone was selected and seeded at a density of 5×10⁴ cells per well in 48 well plates. Luciferase reporter activity was measured in duplicates from extracts of cells after incubating cells in culture medium (DMEM [Gibco-BRL]+10% FCS [PAA laboratories]) for 16 hours (5% CO₂, 37° C.) containing 0,5% DMSO (control) or 0,5% DMSO with increasing concentrations of LN12996.

A dose-dependent transactivation (EC50˜1 μM) of the reporter gene by FXR was observed (FIG. 7). Variations of duplicate measurements are within 20%.

### Example 4

This example illustrates that the compound LN12996 can stimulate the transcription of known endogenous target genes of FXR in human HepG2 hepatoma cells which are transfected with FXR.

Human HepG2 were transfected with the plasmid pCEP-hFXR and a stable clone was selected using hygromycin B by applying standard methods known to those skilled in the art. The plasmid pCEP-hFXR was constructed by placing the coding sequence of human FXR under the transcriptional control of a CMV promoter in the plasmid backbone of pCEP4 (Invitrogen) according to standard proceedures known to those skilled in the art. HepG2-FXR cells were grown in RPMI medium containing 10% FCS (Gibco) in 6 well plates in duplicates and RNA was isolated using the RNAeasy kit from Quiagen according to the manufacturers protocol after growing cells for various time periods in presence of 5 μM of compound LN12996 or DMSO as a control. For the first-strand synthesis the TaqMan Gold RT-PCR Kit (Applied Biosystems) was used and the PCR analysis was done using the TaqMan Universal PCR Master Mix (Appied Biosystems). Probes were labelled with FAM. The analyses were performed using the ABI Prism 7700 Sequence Detection System using the following primers for amplification and detection:

| | |
|---|---|
| BSEP: | |
| Fw: | CTTTCCACTGCAGTGCCATGT |
| Rev: | GCATGGGCACACAATCATTTC |
| Probe (FAM): | CCAATGATGGTATCTGCAGCTCTGACCG |
| Cyp7A: | |
| Fw: | GAGGAACTCAAGAGGATTGGCAC |
| Rev: | GGAATTAGGAGAAGGCAAACGG |
| Probe (FAM): | CAGAGCACAGCCCAGGTATGGAATTAATCC |

The results represented in FIG. 8 A show that the levels of BSEP mRNA are increased after treatment of cells with 5 μM LN12996 for 4, 16 and 36 hours respectively compared to the levels in cells treated with the DMSO control for the same time periods. The levels of Cyp7A mRNA are decreased after treatment of cells with LN12996 compared to the DMSO control at all time points (FIG. 8 B).

### Example 5

To asses the bioavailability of LN12996, rats (n=4 per timepoint) were administered 5 mg/kg by intravenous injection or 10 mg/kg by oral gavage in 5% Carboxymethylcellulose as a vehicle. Blood samples were drawn from jugular and portal veins respectively and plasma concentration of LN12996 was determined by LC-MS-MS according to methods known to those skilled in the art. Serial blood sampling from each rat was performed for I.V. at 0 (predose), 0.018, 0.08, 0.25, 0.5., 1, 2, 4, 6, 8 and 24 h and for P.O. at 0 (predose), 0.018, 0.08, 0.25, 0.5., 1, 2, 4, 6, 8 and 24 h (FIGS. 9 and 10).

### FIGURE CAPTIONS

FIG. 1:

FIG. 1 shows the synthesis of the compounds according to the invention as also described in Example 2.

FIG. 2:

FIG. 2 shows the measurement parameters employed by the Wallace VICTOR2V™ Multilabel Counter which was used for measuring the EC₅₀ values

FIGS. 3 A, B, C AND D:

FIG. 3 A shows SEQ ID NO. 1 which is the protein sequence of the FXR protein a portion of which was used for cloning as described in the examples. FIG. 3 B shows SEQ ID NO. 2 which is the mRNA sequence of the FXR protein. FIG. 3 C shows SEQ ID NO. 3 which is the protein sequence of TIF2 (Acc. No: XM_{—}011633 RefSeq DB), FIG. 3 D shows SEQ ID NO. 4 which is the respective mRNA sequence corresponding to the TIF2 protein.

FIG. 4 A TO B:

FIG. 4 shows the internal molecular name used by the applicant (MOLNAME) as well as the corresponding structures of preferred compounds according to the invention. The figure further shows their respective EC₅₀ values (EC50 AVG) as established according to the experiment 1 n multiple experiments (see above), as well as their respective average efficacy (% activity relative to CDCA control agonist).

FIG. 5:

FIG. 5 shows various known FXR ligands. It is apparent from their structures that the inventors have identified novel compounds which are structurally not related to these known ligands.

FIG. 6 A AND B:

FIG. 6 shows various genes that have been found to be regulated through binding of an FXR agonist to the FXR protein.

FIG. 7 A AND B:

A. The figure shows a dose-dependent transactivation of luciferase activity (EC50˜1 μM) in the HEK293 FXR reporter cell line by compound LN12996 and FXR. B. The figure shows the MOLNAME of compounds and their respective EC₅₀ values as determined in triplicate in the HEK293 FXR reporter cell line (EC50 reporter) as well as their respective relative efficacy in % compared to the GW4064 control agonist (efficacy reporter).

FIG. 8 A AND B:

The figures show dose-dependent influence on the gene expression level determined by TaqMan analysis of known endogenous FXR response genes (A:BSEP; B:Cyp7a) upon administration of cpd LN12996 and the DMSO control in FXR-transfected HepG2 human hepatoma cells.

FIG. 9:

Pharmakokinetic data showing the serum levels reached upon intravenous or oral administration of cpd LN 12996 as an example for a compound which obeys the strucutral requirements of formula (1)

FIG. 10:

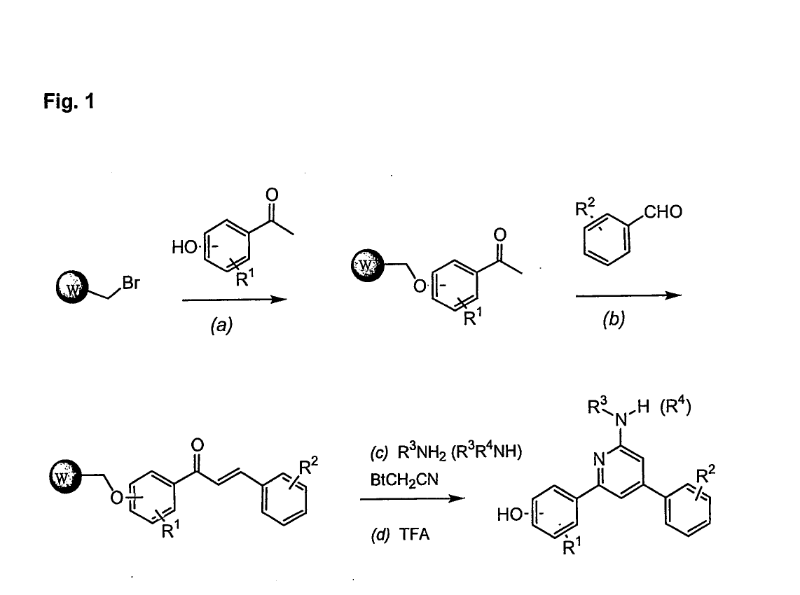

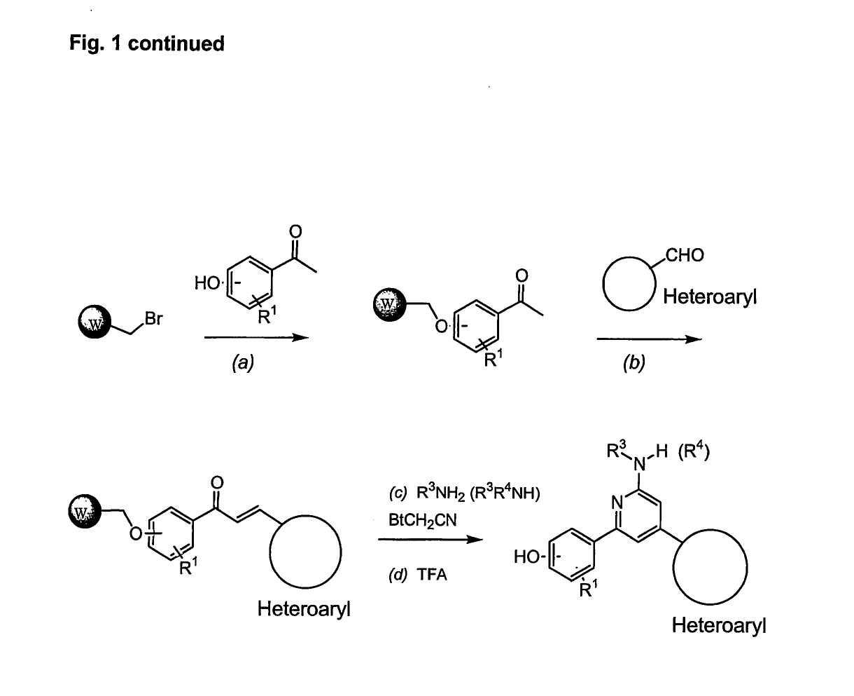

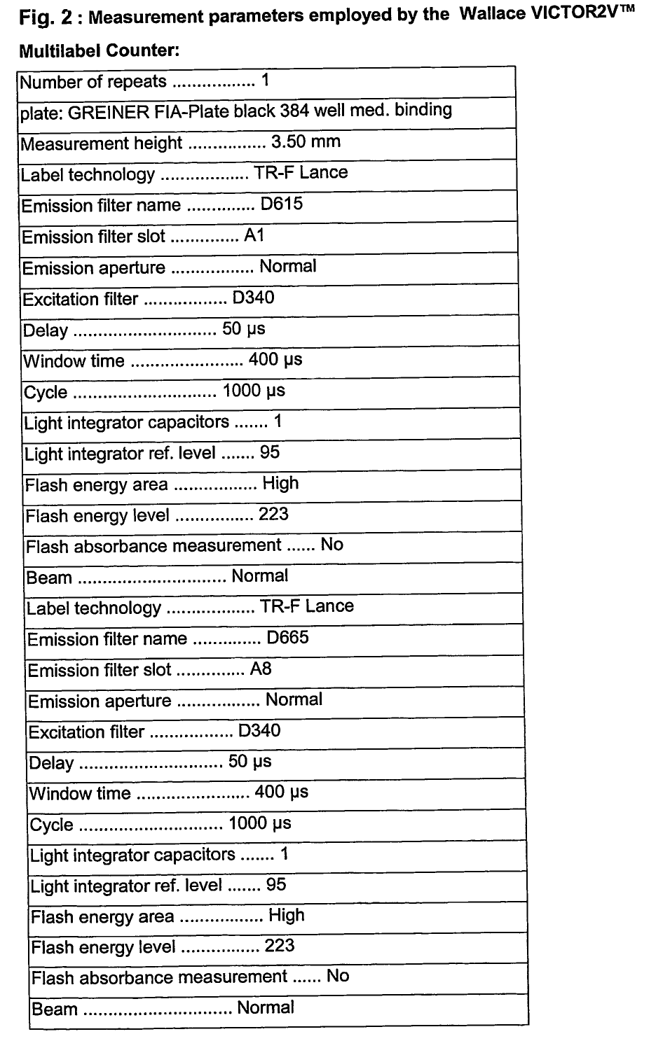

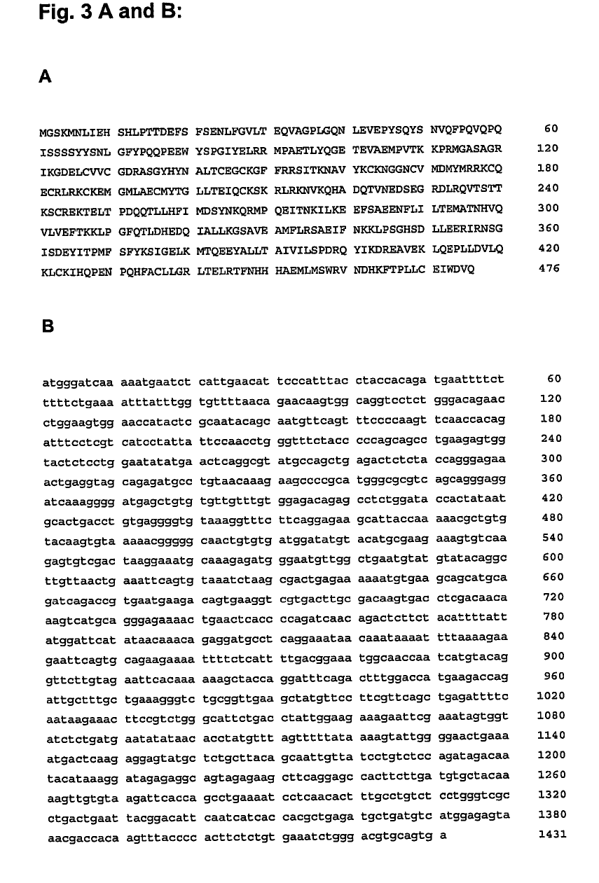

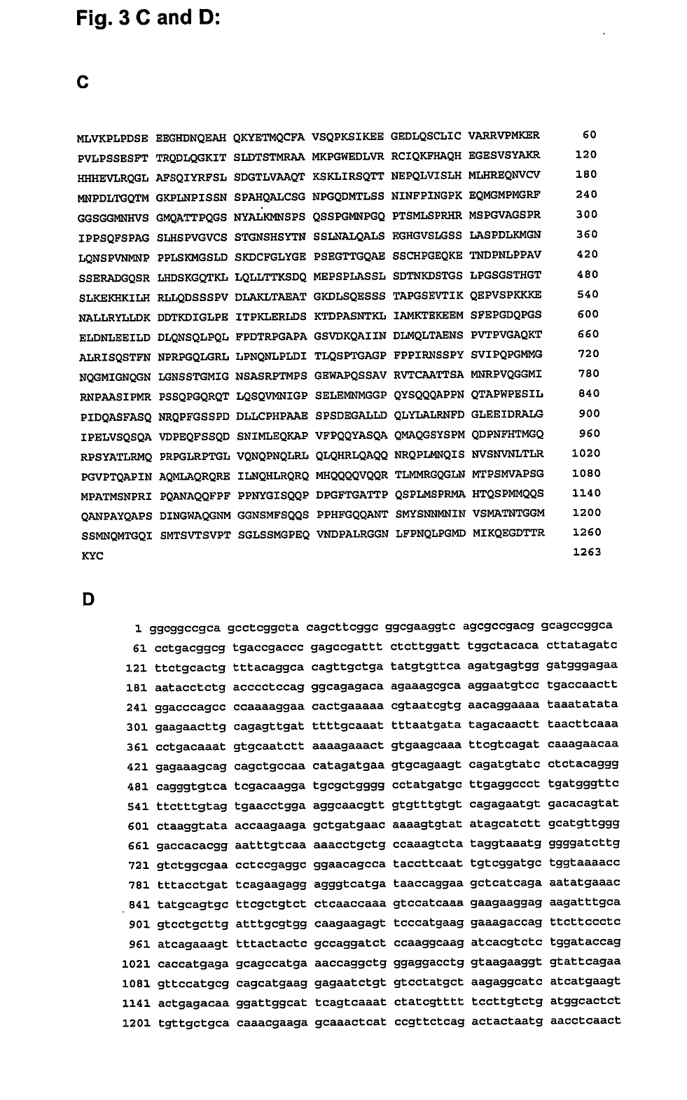

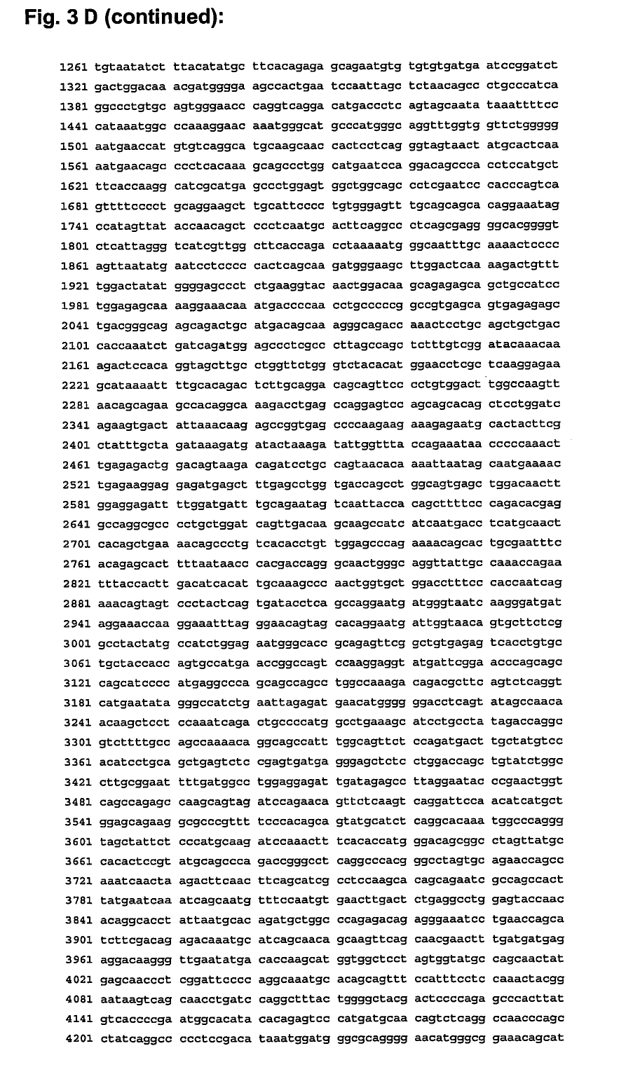

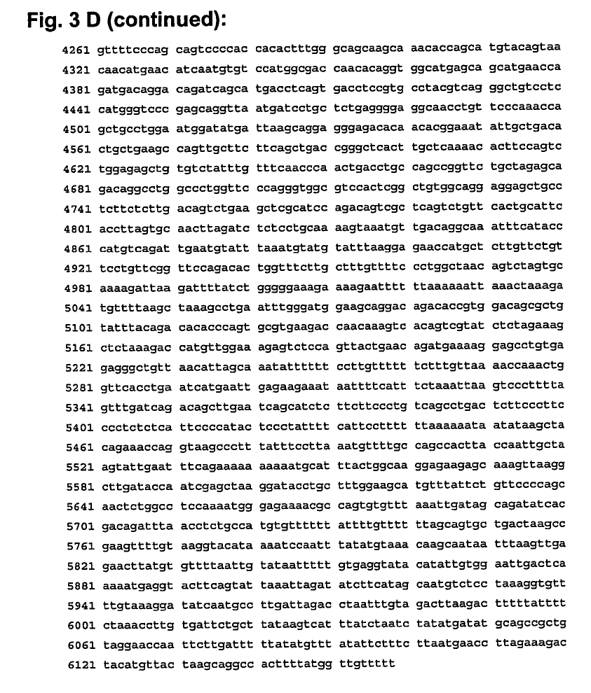

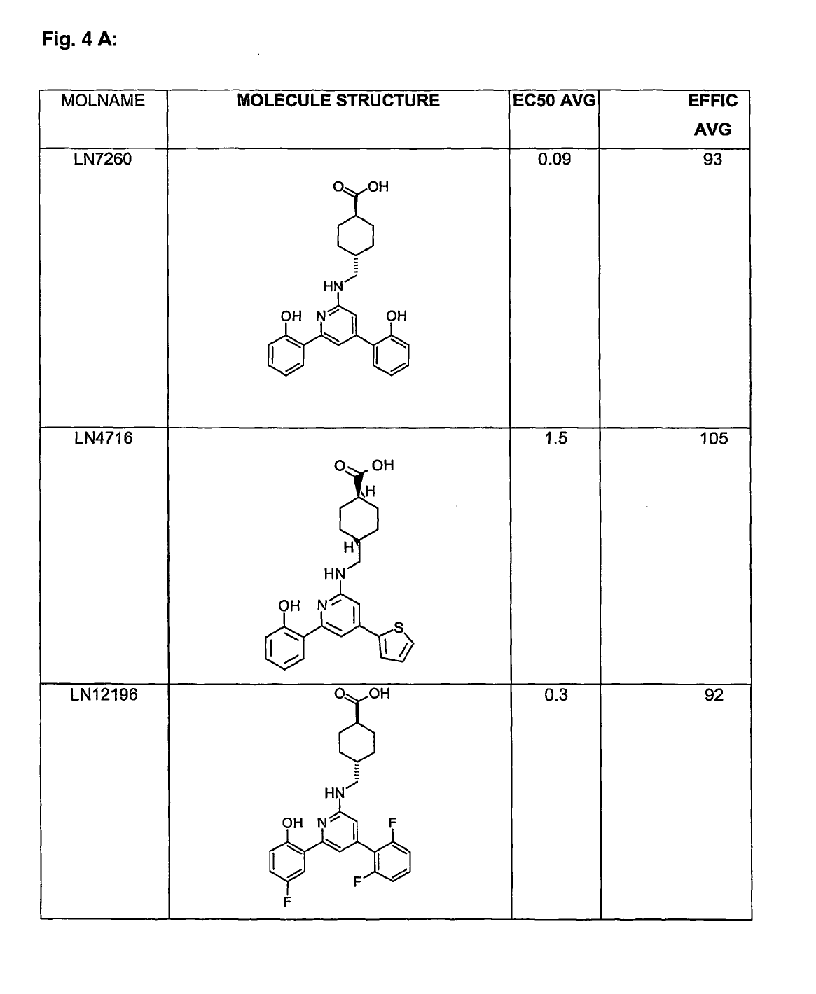

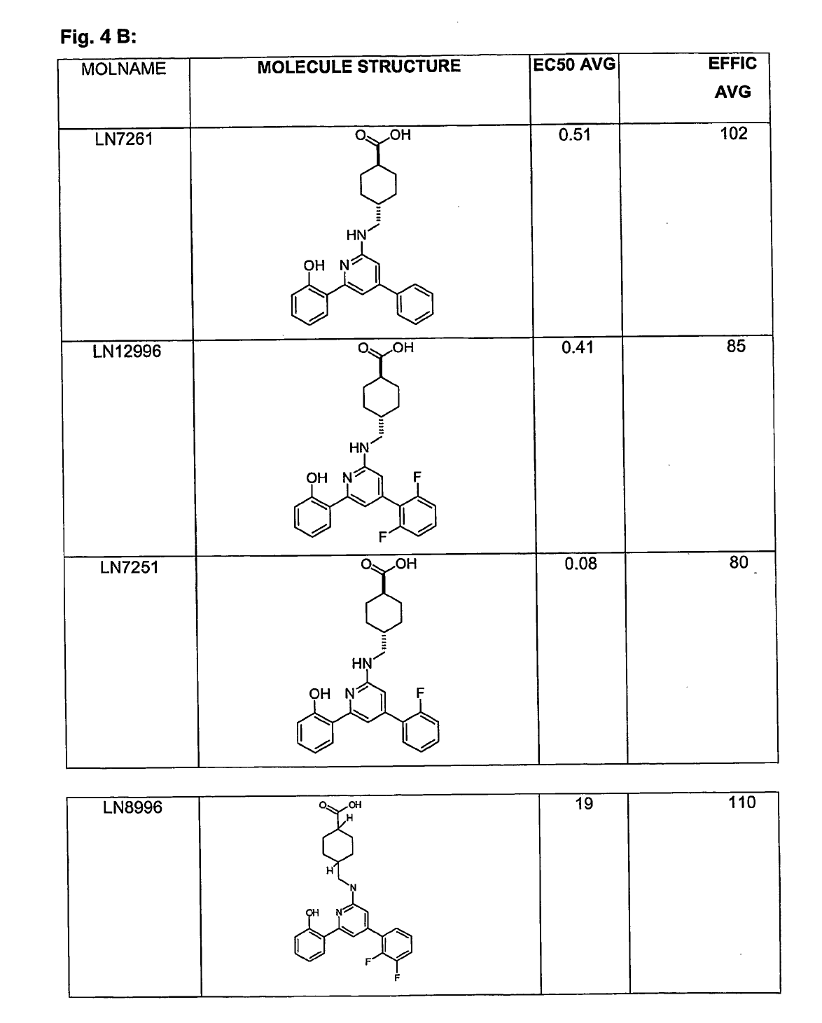

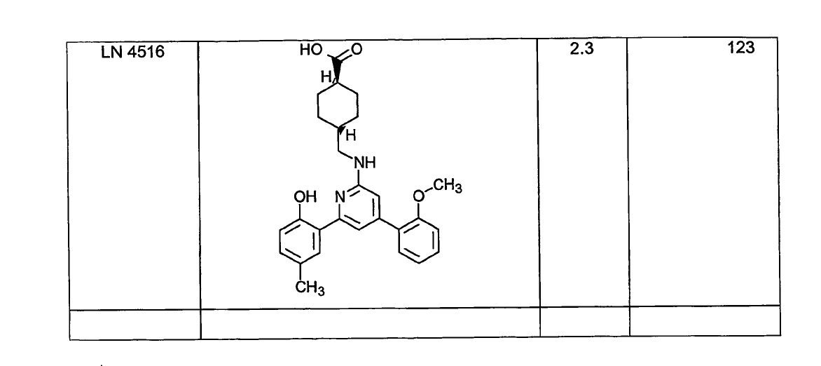

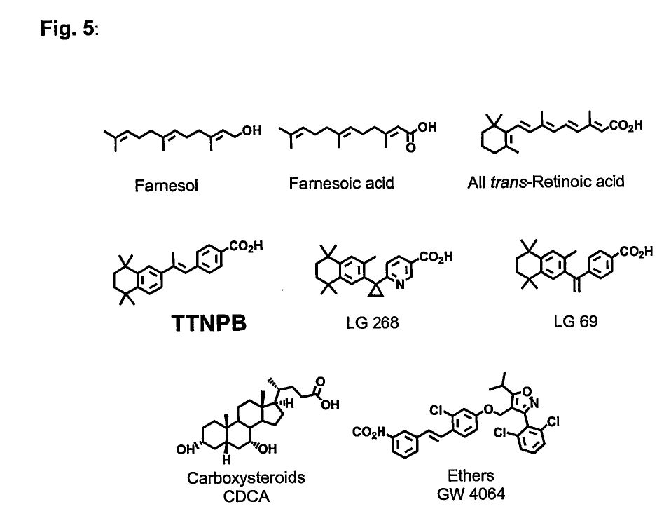

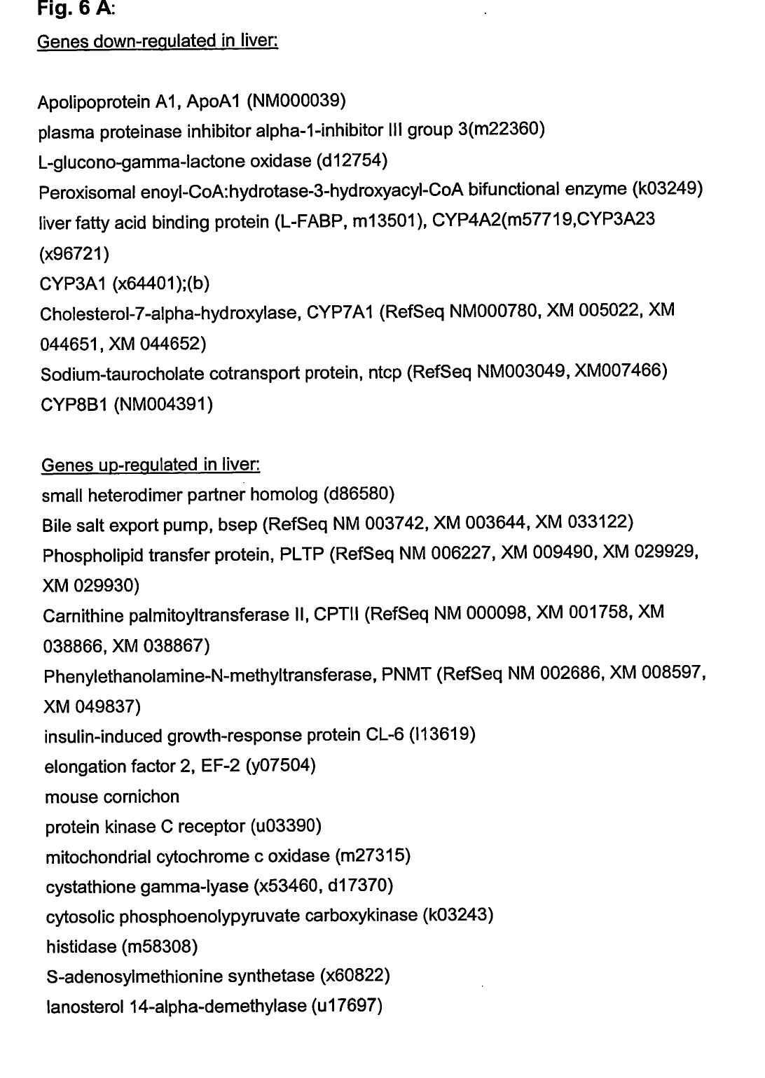

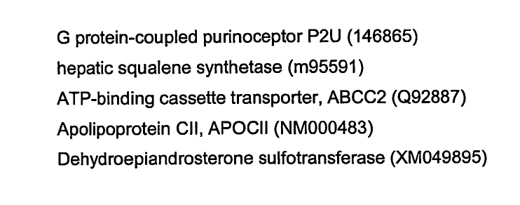

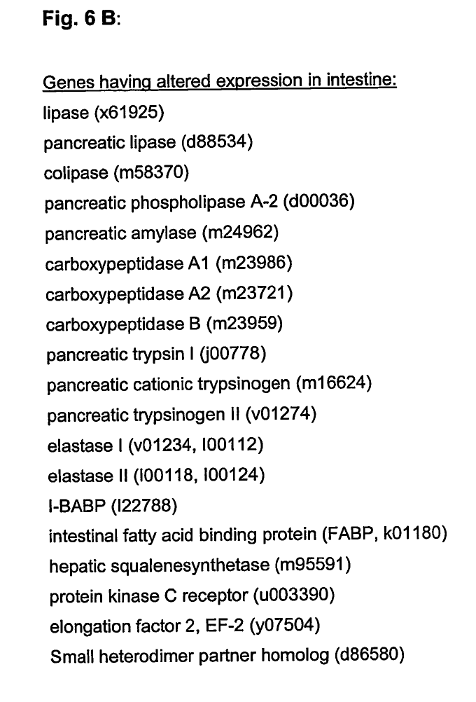

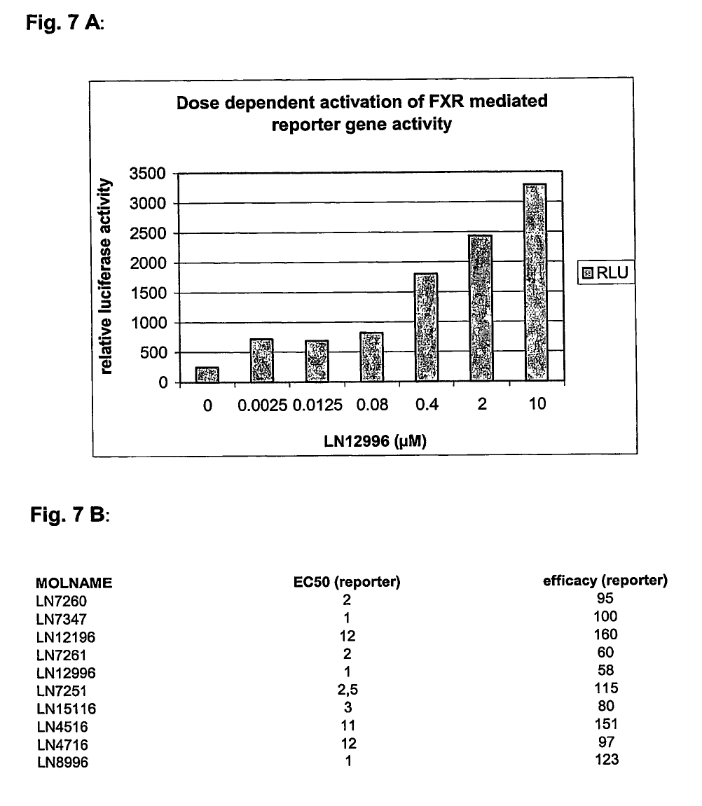

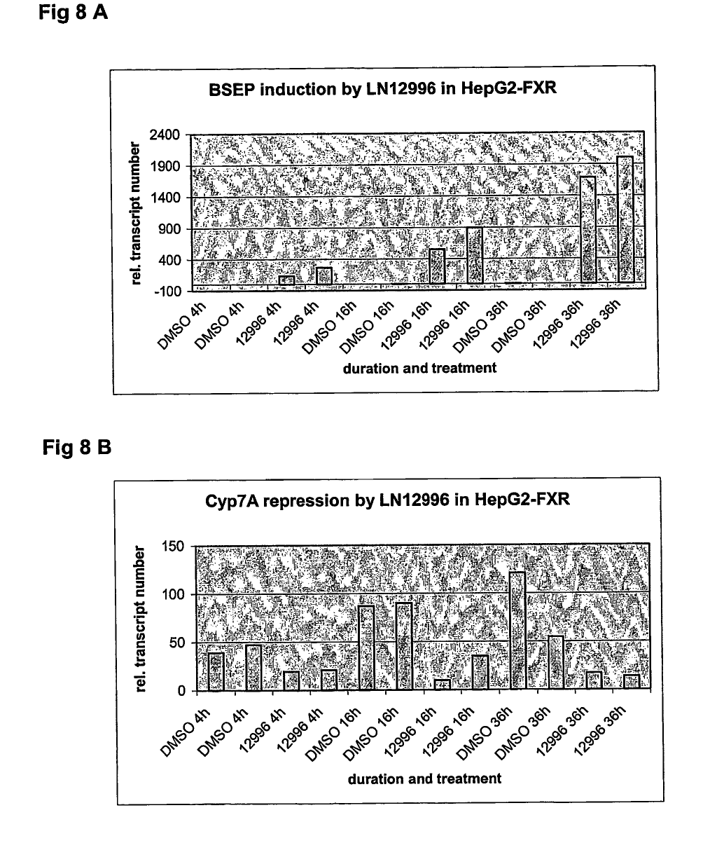

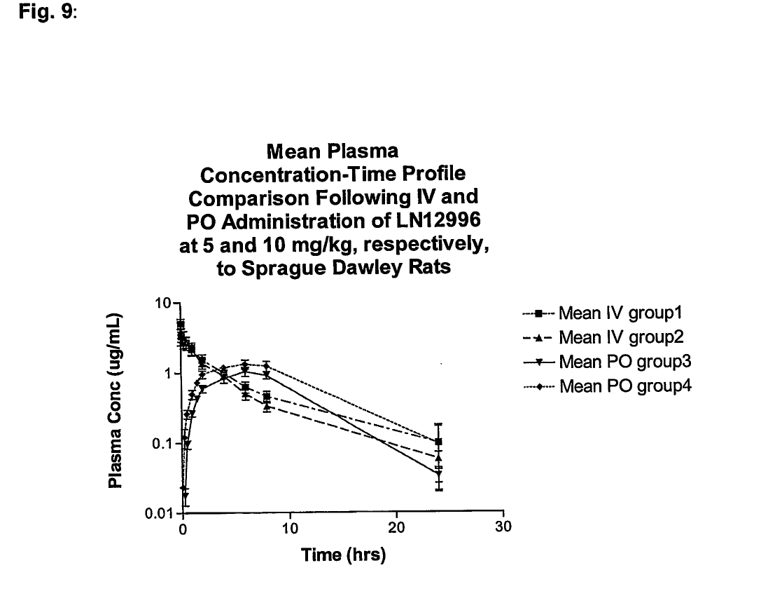

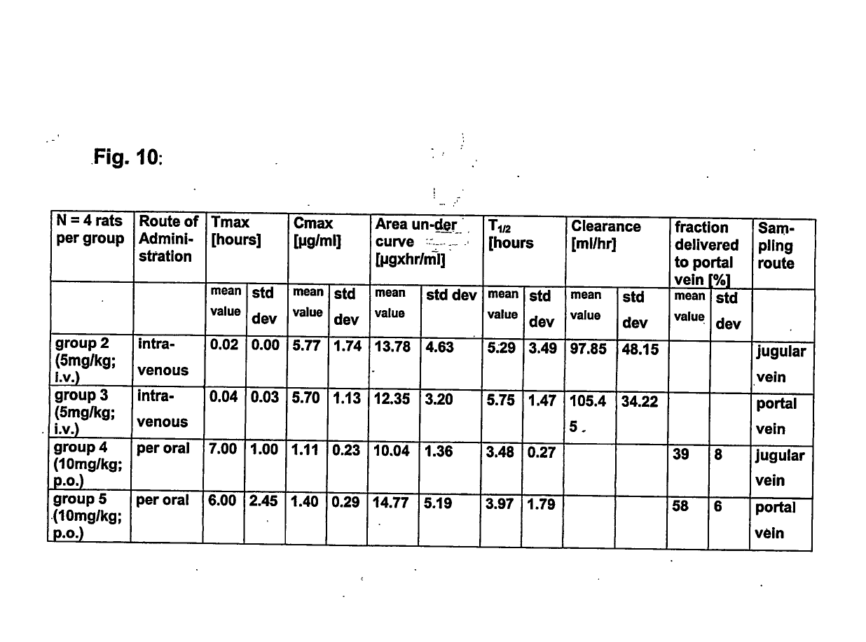

Table which shows maximally reached serum levels and halftime of cpd. LN 12996 after oral or intravenous delivery

The features of the present invention disclosed in the specification, the claims and/or in the accompanying drawings, may, both separately and in any combination thereof, be material for realizing the invention in various forms thereof.

1. A compound including resolved diastereoisomers and enantiomers, and tautomers of the formula (1), or pharmaceutical acceptable salts or solvates thereof, 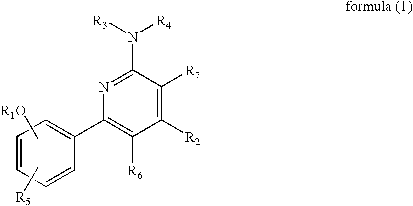
wherein:
R₁ in formula (1) is H, C₁ to C₇ acyl or C₁ to C₇ substituted acyl,
R₂ is phenyl, substituted phenyl, C₅ to C₆ heteroaryl, C₅ to C₆ substituted heteroaryl, naphthyl or substituted naphthyl,
R₃ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl, C₇ to C₁₂ substituted phenylalkyl, or phenyl,
R₄ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl or C₇ to C₁₂ substituted phenylalkyl,
R₃ and R₄ may be taken together with nitrogen to form a heterocycle or substituted heterocycle, or a heteroaryl or substituted heteroaryl ring,
R₅ is H, C₁ to C₈ alkyl, halogen, hydroxy, alkoxy, in particular C₁ to C₈ alkoxy, carboxy, ester, amide or C₁ to C₈ aminoacyl,
R₆ is H, C₁ to C₈ alkyl, C₁ to C₈ substituted alkyl and
R₇ is H, F, Cl, methyl, or trifluoromethyl.

2. The compound according to claim 1, or pharmaceutical acceptable salts or solvates thereof, wherein:
R₁ is H,
R₂ is substituted phenyl, C₅ to C₆ heteroaryl, or substituted C₅ to C₆ heteroaryl,
R₃ is H,
R₄ in formula (1) is a structure according to formula (2),
—CH₂—R₄₋₁—CO₂R₈, formula (2)
wherein:
R₄₋₁ is C₁ to C₈ alkyl, C₁ to C₈ substituted alkyl, C₃ to C₈ cycloalkyl, C₃ to C₈ substituted cycloalkyl, C₅ to C₆ aryl, C₅ to C₆ substituted aryl, C₅ to C₆ heteroaryl, or C₅ to C₆ substituted heteroaryl,
and the methylene and the COOR₈ substituents take the [1,4]-positions in case R₄₋₁ is cyclohexyl, substituted cyclohexyl, C₆ aryl, C₆ substituted aryl, C₆ heteroaryl, or C₆ substituted heteroaryl; or the methylene and the COOR₈-substituents take the [1,3]-positions in case R₄₋₁ is cyclopentyl, substituted cyclopentyl, or substituted C₅ heteroaryl; the methylene and the COOR₈ substituents can have all possible diastereomeric configurations,
R₅ is H, a halogen, hydroxy, alkoxy or C₁ to C₈ alkyl,
R₆ is H,
R₇ is H, and
R₈ is H, methyl or ethyl.

3. The compound according to claim 1, or pharmaceutical acceptable salts or solvates thereof, wherein:
R₃ is H,
R₄ in formula (1) is a structure according to formula (3),
—R₄₋₁—CO₂R₈, formula (3)
wherein:
R₄₋₁ is C₁ to C₈ alkyl, C₁ to C₈ substituted alkyl, C₃ to C₈ cycloalkyl, C₃ to C₈ substituted cycloalkyl, C₅ to C₆ aryl, C₅ to C₆ substituted aryl, C₅ to C₆ heteroaryl, or C₅ to C₆ substituted heteroaryl,
and the methylene and the COOR₈ substituents take the [1,4]-positions in case R₄₋₁ is cyclohexyl, substituted cyclohexyl, C₆ aryl, C₆ substituted aryl, C₆ heteroaryl, or C₆ substituted heteroaryl; or the methylene and the COOR₈-substituents take the [1,3]-positions in case R₄₋₁ is cyclopentyl, substituted cyclopentyl, or substituted C₅ heteroaryl; the methylene and the COOR₈ substituents can have all possible diastereomeric configurations.

4. The compound according to claim 1, or pharmaceutical acceptable salts or solvates thereof, wherein:
R₁ is H,
R₂ is substituted phenyl,
R₃ is H,
R₄ in formula (1) is a structure according to formula (4), 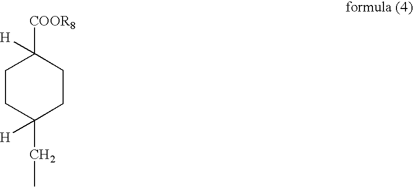
wherein the methylene and the COOR₈ substituents can have all possible diastereomeric configurations,
R₅ is H, a halogen, hydroxy, alkoxy or C₁ to C₈ alkyl,
R₆ is H,
R₇ is H, and
R₈ is H, methyl or ethyl.

5. The compound according to claim 1, of the formula (5), or pharmaceutical acceptable salts or solvates thereof, 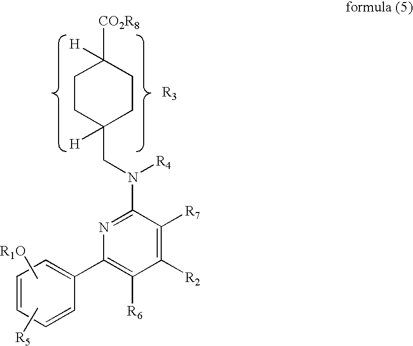
wherein:
R₁ is H,
R₂ is substituted phenyl, C₅ to C₆ heteroaryl, or C₅ to C₆ substituted heteroaryl,
R₄ is H and R₃ is a structure according to formula (6) 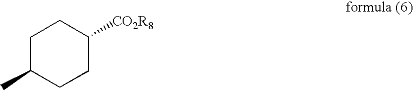
wherein the COOR₈ and the methylene substituents are in double axial (a,a) positions,
R₅ is H, a halogen, hydroxy, alkoxy or C₁ to C₈ alkyl,
R₆ is H,
R₇ is H, and
R₈ is H, methyl or ethyl.

6. The compound according to claim 1, of the formula (7)

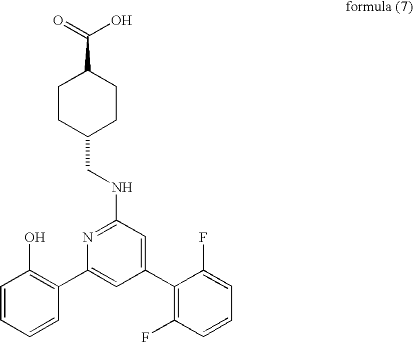

7. The compound according to claim 1, of the formula (8)

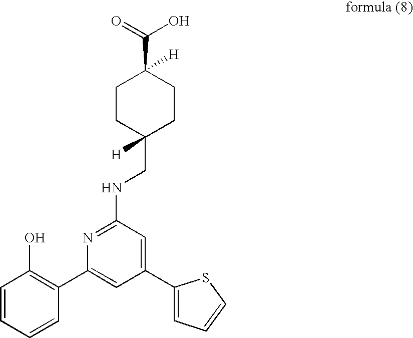

8. The compound according to claim 1, of the formula (9)

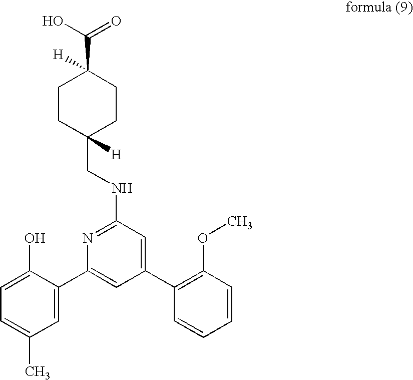

9. The compound according to claim 1, of the formula (10)

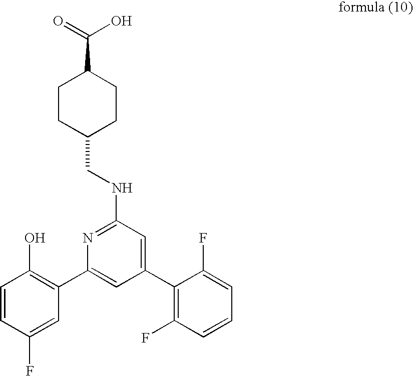

10. The compound according to claim 1, of the formula (11)

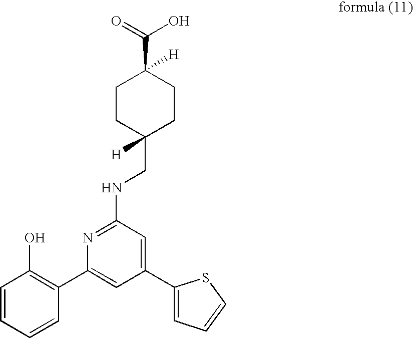

11. The compound according to claim 1, of the formula (12)

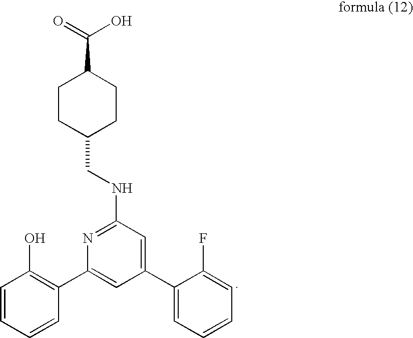

12. The compound according to claim 1, of the formula (13)

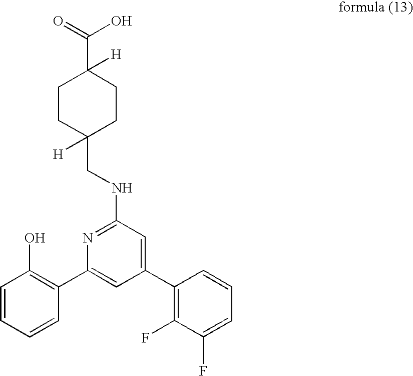

13. The compound according to claim 1, of the formula (14)

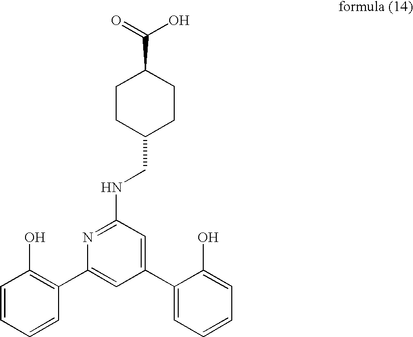

14. (canceled)

15. The compound according to claim 1, wherein said compound binds with the human NR1H4 receptor protein or a portion thereof or a mammalian homologue thereof according to SEQ ID NO. 1.

16-25. (canceled)

26. The method, according to claim 36, which is for the prevention or treatment of a disease or condition which is mediated or can be addressed by the NR1H4 receptor in a mammal.

27. The method, according to claim 26, which is for regulating bile flow or the bile acid transport system in a mammal.

28. The method, according to claim 36, which is for treating in a mammal a disease or condition which is affected by impaired blood levels of cholesterol, lipoproteins, phospholipids, triglycerides, or bile acids and/or impaired bile flow or impaired bile levels of cholesterol, phospholipids or bile acids.

29. The method, according to claim 36, which is for treating in a mammal cholestatic conditions.

30. A method for treating in a mammal malign proliferative diseases such as cancer which can be treated by inducing apoptosis in the affected cells or tissues comprising administering to a mammal in need of such treatment a therapeutically effective amount of a compound including resolved diastereoisomers and enantiomers, and tautomers of the formula (1), or pharmaceutical acceptable salts or solvates thereof, 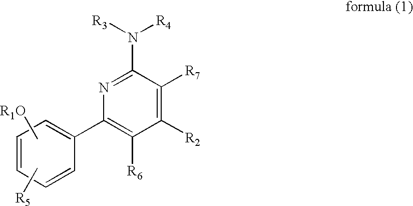
wherein:
R₁ in formula (1) is H, C₁ to C₇ acyl or C₁ to C₇ substituted acyl,
R₂ is phenyl, substituted phenyl, C₅ to C₆ heteroaryl, C₅ to C₆ substituted heteroaryl, naphthyl or substituted naphthyl,
R₃ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl, C₇ to C₁₂ substituted phenylalkyl, or phenyl,
R₄ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl or C₇ to C₁₂ substituted phenylalkyl,
R₃ and R₄ may be taken together with nitrogen to form a heterocycle or substituted heterocycle, or a heteroaryl or substituted heteroaryl ring,
R₅ is H, C₁ to C₈ alkyl, halogen, hydroxy, alkoxy, in particular C₁ to C₈ alkoxy, carboxy, ester, amide or C₁ to C₈ aminoacyl,
R₆ is H, C₁ to C₈ alkyl, C₁ to C₈ substituted alkyl and
R₇ is H, F, Cl, methyl, or trifluoromethyl.

31. A method for treating in a mammal conditions of drug resistance that arise during drug treatment of disorders such as cancer or infectious diseases, or during continuous administration of contraceptive drugs comprising administering to a mammal in need of such treatment a therapeutically effective amount of a compound including resolved diastereoisomers and enantiomers, and tautomers of the formula (1), or pharmaceutical acceptable salts or solvates thereof, 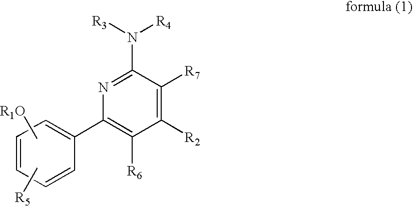
wherein:
R₁ in formula (1) is H, C₁ to C₇ acyl or C₁ to C₇ substituted acyl,
R₂ is phenyl, substituted phenyl, C₅ to C₆ heteroaryl, C₅ to C₆ substituted heteroaryl, naphthyl or substituted naphthyl,
R₃ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl, C₇ to C₁₂ substituted phenylalkyl, or phenyl,
R₄ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl or C₇ to C₁₂ substituted phenylalkyl,
R₃ and R₄ may be taken together with nitrogen to form a heterocycle or substituted heterocycle, or a heteroaryl or substituted heteroaryl ring,
R₅ is H, C₁ to C₈ alkyl, halogen, hydroxy, alkoxy, in particular C₁ to C₈ alkoxy, carboxy, ester, amide or C₁ to C₈ aminoacyl,
R₆ is H, C₁ to C₈ alkyl, C₁ to C₈ substituted alkyl and
R₇ is H, F, Cl, methyl, or trifluoromethyl.

32. The method, according to claim 36, which is for blocking in a mammal the cholesterol absorption or bile acid re-absorption in the intestine of a mammal in need of such blocking.

33. A method for regulating the expression of NR1H4 responsive genes wherein said method comprises administering a therapeutically effective amount of a compound including resolved diastereoisomers and enantiomers, and tautomers of the formula (1), or pharmaceutical acceptable salts or solvates thereof, 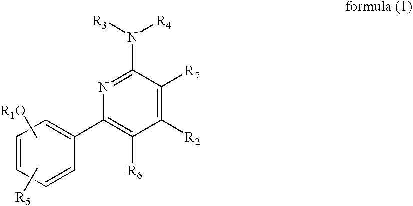
wherein:
R₁ in formula (1) is H, C₁ to C₇ acyl or C₁ to C₇ substituted acyl,
R₂ is phenyl, substituted phenyl, C₅ to C₆ heteroaryl, C₅ to C₆ substituted heteroaryl, naphthyl or substituted naphthyl,
R₃ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl, C₇ to C₁₂ substituted phenylalkyl, or phenyl,
R₄ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl or C₇ to C₁₂ substituted phenylalkyl,
R₃ and R₄ may be taken together with nitrogen to form a heterocycle or substituted heterocycle, or a heteroaryl or substituted heteroaryl ring,
R₅ is H, C₁ to C₈ alkyl, halogen, hydroxy, alkoxy, in particular C₁ to C₈ alkoxy, carboxy, ester, amide or C₁ to C₈ aminoacyl,
R₆ is H, C₁ to C₈ alkyl, C₁ to C₈ substituted alkyl and
R₇ is H, F, Cl, methyl, or trifluoromethyl.

34. The method, according to claim 36, which is for modulating the expression of the intestinal bile acid binding protein (IBABP) in intestinal mucosa cells and/or cholangiocytes by the NR1H4 receptor in a mammal.

35. A method according to claim 36 where the mammal is a human.

36. A method for providing treatment to a mammal in need of one or more of the following:
a) regulation of bile flow or the bile acid transport system by activating or repressing the NR1H4 receptor;
b) treating a disease or condition which is affected by impaired blood levels of cholesterol, lipoproteins, phospholipids, triglycerides, or bile acids and/or impaired bile flow or impaired bile levels of cholesterol, phospholipids or bile acids;
c) treating cholestatic conditions;
d) blocking the cholesterol absorption or bile acid re-absorption in the intestine of the mammal; and
e) modulating the expression of the intestinal bile acid binding protein (IBABP) in intestinal mucosa cells and/or cholangiocytes by the NR1H4 receptor;
wherein said method comprises administering, to the mammal, a therapeutically effective amount of a compound including resolved diastereoisomers and enantiomers, and tautomers of the formula (1), or pharmaceutical acceptable salts or solvates thereof, 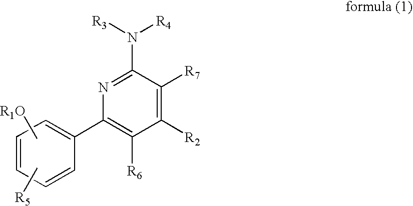
wherein:
R₁ in formula (1) is H, C₁ to C₇ acyl or C₁ to C₇ substituted acyl,
R₂ is phenyl, substituted phenyl, C₅ to C₆ heteroaryl, C₅ to C₆ substituted heteroaryl, naphthyl or substituted naphthyl,
R₃ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl, C₇ to C₁₂ substituted phenylalkyl, or phenyl,
R₄ is H, C₁ to C₈ alkyl, C₁ to C₈ substituted alkyl, C₃ to C₈ cycloalkyl, C₃ to C₈ substituted cycloalkyl, C₇ to C₁₂ alkylphenyl or C₇ to C₁₂ substituted phenylalkyl,
R₃ and R₄ may be taken together with nitrogen to form a heterocycle or substituted heterocycle, or a heteroaryl or substituted heteroaryl ring,
R₅ is H, C₁ to C₈ alkyl, halogen, hydroxy, alkoxy, in particular C₁ to C₈ alkoxy, carboxy, ester, amide or C₁ to C₈ aminoacyl,
R₆ is H, C₁ to C₈ alkyl, C₁ to C₈ substituted alkyl and
R₇ is H, F, Cl, methyl, or trifluoromethyl.

37. The method, according to claim 29, wherein said condition is selected from the group consisting of primary biliary cirrhosis (PBC), progressive familiary cholestasis (PFIC), estrogen or drug induced cholestasis, extrahepatic cholestasis, secondary forms of cholestasis, atherosclerosis, gallstone disease, lipid disorders, obesity, and cardiovascular and metabolic disorders.

38. The method, according to claim 33, wherein said gene is selected from genes that encode cholesterol-7-alpha hydroxylase (cyp7a1), sterol-12-alpha hydroxylase (cyp8b1), small heterodimer partner (shp), phospholipid transfer protein (pltp), bile salt export pump (bsep), sodium-taurocholate co-transporter (ntcp), organic anion transport proteins 1 and 2 (oatp1 and -2), canalicular multidrug resistance protein 2 (mdr2) or other genes that are members of the cytochrom P450 family or members of the ABC-transporter family or members of the MDR class III multidrug resistance proteins or members of the MRP multidrug resistance protein family or members of the nuclear receptor gene family.

39. The method, according to claim 30, wherein said mammal is a human.

40. The method, according to claim 31, wherein said mammal is a human.

41. The method, according to claim 33, wherein said mammal is a human.


SEQUENCE LISTING (5 sequences)

SEQ ID NO: 1 - protein, 476 aa, Homo sapiens
MGSKMNLIEHSHLPTTDEFSFSENLFGVLTEQVAGPLGQNLEVEPYSQYSNVQFPQVQPQISSSSYYSNLGFYPQQPEEWYSPGIYELRRMPAETLYQGETEVAEMPVTKKPRMGASAGRIKGDELCVVCGDRASGYHYNALTCEGCKGFFRRSITKNAVYKCKNGGNCVMDMYMRRKCQECRLRKCKEMGMLAECMYTGLLTEIQCKSKRLRKNVKQHADQTVNEDSEGRDLRQVTSTTKSCREKTELTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVQ

SEQ ID NO: 2 - DNA, 1431 bp, Homo sapiens
atgggatcaaaaatgaatctcattgaacattcccatttacctaccacagatgaattttctttttctgaaaatttatttggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgtatgtatacaggcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtga

SEQ ID NO: 3 - protein, 1263 aa, Homo sapiens
MLVKPLPDSEEEGHDNQEAHQKYETMQCFAVSQPKSIKEEGEDLQSCLICVARRVPMKERPVLPSSESFTTRQDLQGKITSLDTSTMRAAMKPGWEDLVRRCIQKFHAQHEGESVSYAKRHHHEVLRQGLAFSQIYRFSLSDGTLVAAQTKSKLIRSQTTNEPQLVISLHMLHREQNVCVMNPDLTGQTMGKPLNPISSNSPAHQALCSGNPGQDMTLSSNINFPINGPKEQMGMPMGRFGGSGGMNHVSGMQATTPQGSNYALKMNSPSQSSPGMNPGQPTSMLSPRHRMSPGVAGSPRIPPSQFSPAGSLHSPVGVCSSTGNSHSYTNSSLNALQALSEGHGVSLGSSLASPDLKMGNLQNSPVNMNPPPLSKMGSLDSKDCFGLYGEPSEGTTGQAESSCHPGEQKETNDPNLPPAVSSERADGQSRLHDSKGQTKLLQLLTTKSDQMEPSPLASSLSDTNKDSTGSLPGSGSTHGTSLKEKHKILHRLLQDSSSPVDLAKLTAEATGKDLSQESSSTAPGSEVTIKQEPVSPKKKENALLRYLLDKDDTKDIGLPEITPKLERLDSKTDPASNTKLIAMKTEKEEMSFEPGDQPGSELDNLEEILDDLQNSQLPQLFPDTRPGAPAGSVDKQAIINDLMQLTAENSPVTPVGAQKTALRISQSTFNNPRPGQLGRLLPNQNLPLDITLQSPTGAGPFPPIRNSSPYSVIPQPGMMGNQGMIGNQGNLGNSSTGMIGNSASRPTMPSGEWAPQSSAVRVTCAATTSAMNRPVQGGMIRNPAASIPMRPSSQPGQRQTLQSQVMNIGPSELEMNMGGPQYSQQQAPPNQTAPWPESILPIDQASFASQNRQPFGSSPDDLLCPHPAAESPSDEGALLDQLYLALRNFDGLEEIDRALGIPELVSQSQAVDPEQFSSQDSNIMLEQKAPVFPQQYASQAQMAQGSYSPMQDPNFHTMGQRPSYATLRMQPRPGLRPTGLVQNQPNQLRLQLQHRLQAQQNRQPLMNQISNVSNVNLTLRPGVPTQAPINAQMLAQRQREILNQHLRQRQMHQQQQVQQRTLMMRGQGLNMTPSMVAPSGMPATMSNPRIPQANAQQFPFPPNYGISQQPDPGFTGATTPQSPLMSPRMAHTQSPMMQQSQANPAYQAPSDINGWAQGNMGGNSMFSQQSPPHFGQQANTSMYSNNMNINVSMATNTGGMSSMNQMTGQISMTSVTSVPTSGLSSMGPEQVNDPALRGGNLFPNQLPGMDMIKQEGDTTRKYC

SEQ ID NO: 4 - DNA, 6158 bp, Homo sapiens
ggcggccgcagcctcggctacagcttcggcggcgaaggtcagcgccgacggcagccggcacctgacggcgtgaccgacccgagccgatttctcttggatttggctacacacttatagatcttctgcactgtttacaggcacagttgctgatatgtgttcaagatgagtgggatgggagaaaatacctctgacccctccagggcagagacaagaaagcgcaaggaatgtcctgaccaacttggacccagccccaaaaggaacactgaaaaacgtaatcgtgaacaggaaaataaatatatagaagaacttgcagagttgatttttgcaaattttaatgatatagacaactttaacttcaaacctgacaaatgtgcaatcttaaaagaaactgtgaagcaaattcgtcagatcaaagaacaagagaaagcagcagctgccaacatagatgaagtgcagaagtcagatgtatcctctacagggcagggtgtcatcgacaaggatgcgctggggcctatgatgcttgaggcccttgatgggttcttctttgtagtgaacctggaaggcaacgttgtgtttgtgtcagagaatgtgacacagtatctaaggtataaccaagaagagctgatgaacaaaagtgtatatagcatcttgcatgttggggaccacacggaatttgtcaaaaacctgctgccaaagtctataggtaaatgggggatcttggtctggcgaacctccgaggcggaacagccataccttcaattgtcggatgctggtaaaacctttacctgattcagaagaggagggtcatgataaccaggaagctcatcagaaatatgaaactatgcagtgcttcgctgtctctcaaccaaagtccatcaaagaagaaggagaagatttgcagtcctgcttgatttgcgtggcaagaagagttcccatgaaggaaagaccagttcttccctcatcagaaagttttactactcgccaggatctccaaggcaagatcacgtctctggataccagcaccatgagagcagccatgaaaccaggctgggaggacctggtaagaaggtgtattcagaagttccatgcgcagcatgaaggagaatctgtgtcctatgctaagaggcatcatcatgaagtactgagacaaggattggcattcagtcaaatctatcgtttttccttgtctgatggcactcttgttgctgcacaaacgaagagcaaactcatccgttctcagactactaatgaacctcaacttgtaatatctttacatatgcttcacagagagcagaatgtgtgtgtgatgaatccggatctgactggacaaacgatggggaagccactgaatccaattagctctaacagccctgcccatcaggccctgtgcagtgggaacccaggtcaggacatgaccctcagtagcaatataaattttcccataaatggcccaaaggaacaaatgggcatgcccatgggcaggtttggtggttctgggggaatgaaccatgtgtcaggcatgcaagcaaccactcctcagggtagtaactatgcactcaaaatgaacagcccctcacaaagcagccctggcatgaatccaggacagcccacctccatgctttcaccaaggcatcgcatgagccctggagtggctggcagccctcgaatcccacccagtcagttttcccctgcaggaagcttgcattcccctgtgggagtttgcagcagcacaggaaatagccatagttataccaacagctccctcaatgcacttcaggccctcagcgaggggcacggggtctcattagggtcatcgttggcttcaccagacctaaaaatgggcaatttgcaaaactccccagttaatatgaatcctcccccactcagcaagatgggaagcttggactcaaaagactgttttggactatatggggagccctctgaaggtacaactggacaagcagagagcagctgccatcctggagagcaaaaggaaacaaatgaccccaacctgcccccggccgtgagcagtgagagagctgacgggcagagcagactgcatgacagcaaagggcagaccaaactcctgcagctgctgaccaccaaatctgatcagatggagccctcgcccttagccagctctttgtcggatacaaacaaagactccacaggtagcttgcctggttctgggtctacacatggaacctcgctcaaggagaagcataaaattttgcacagactcttgcaggacagcagttcccctgtggacttggccaagttaacagcagaagccacaggcaaagacctgagccaggagtccagcagcacagctcctggatcagaagtgactattaaacaagagccggtgagccccaagaagaaagagaatgcactacttcgctatttgctagataaagatgatactaaagatattggtttaccagaaataacccccaaacttgagagactggacagtaagacagatcctgccagtaacacaaaattaatagcaatgaaaactgagaaggaggagatgagctttgagcctggtgaccagcctggcagtgagctggacaacttggaggagattttggatgatttgcagaatagtcaattaccacagcttttcccagacacgaggccaggcgcccctgctggatcagttgacaagcaagccatcatcaatgacctcatgcaactcacagctgaaaacagccctgtcacacctgttggagcccagaaaacagcactgcgaatttcacagagcacttttaataacccacgaccagggcaactgggcaggttattgccaaaccagaatttaccacttgacatcacattgcaaagcccaactggtgctggacctttcccaccaatcagaaacagtagtccctactcagtgatacctcagccaggaatgatgggtaatcaagggatgataggaaaccaaggaaatttagggaacagtagcacaggaatgattggtaacagtgcttctcggcctactatgccatctggagaatgggcaccgcagagttcggctgtgagagtcacctgtgctgctaccaccagtgccatgaaccggccagtccaaggaggtatgattcggaacccagcagccagcatccccatgaggcccagcagccagcctggccaaagacagacgcttcagtctcaggtcatgaatatagggccatctgaattagagatgaacatggggggacctcagtatagccaacaacaagctcctccaaatcagactgccccatggcctgaaagcatcctgcctatagaccaggcgtcttttgccagccaaaacaggcagccatttggcagttctccagatgacttgctatgtccacatcctgcagctgagtctccgagtgatgagggagctctcctggaccagctgtatctggccttgcggaattttgatggcctggaggagattgatagagccttaggaatacccgaactggtcagccagagccaagcagtagatccagaacagttctcaagtcaggattccaacatcatgctggagcagaaggcgcccgttttcccacagcagtatgcatctcaggcacaaatggcccagggtagctattctcccatgcaagatccaaactttcacaccatgggacagcggcctagttatgccacactccgtatgcagcccagaccgggcctcaggcccacgggcctagtgcagaaccagccaaatcaactaagacttcaacttcagcatcgcctccaagcacagcagaatcgccagccacttatgaatcaaatcagcaatgtttccaatgtgaacttgactctgaggcctggagtaccaacacaggcacctattaatgcacagatgctggcccagagacagagggaaatcctgaaccagcatcttcgacagagacaaatgcatcagcaacagcaagttcagcaacgaactttgatgatgagaggacaagggttgaatatgacaccaagcatggtggctcctagtggtatgccagcaactatgagcaaccctcggattccccaggcaaatgcacagcagtttccatttcctccaaactacggaataagtcagcaacctgatccaggctttactggggctacgactccccagagcccacttatgtcaccccgaatggcacatacacagagtcccatgatgcaacagtctcaggccaacccagcctatcaggccccctccgacataaatggatgggcgcaggggaacatgggcggaaacagcatgttttcccagcagtccccaccacactttgggcagcaagcaaacaccagcatgtacagtaacaacatgaacatcaatgtgtccatggcgaccaacacaggtggcatgagcagcatgaaccagatgacaggacagatcagcatgacctcagtgacctccgtgcctacgtcagggctgtcctccatgggtcccgagcaggttaatgatcctgctctgaggggaggcaacctgttcccaaaccagctgcctggaatggatatgattaagcaggagggagacacaacacggaaatattgctgacactgctgaagccagttgcttcttcagctgaccgggctcacttgctcaaaacacttccagtctggagagctgtgtctatttgtttcaacccaactgacctgccagccggttctgctagagcagacaggcctggccctggttcccagggtggcgtccactcggctgtggcaggaggagctgcctcttctcttgacagtctgaagctcgcatccagacagtcgctcagtctgttcactgcattcaccttagtgcaacttagatctctcctgcaaaagtaaatgttgacaggcaaatttcatacccatgtcagattgaatgtatttaaatgtatgtatttaaggagaaccatgctcttgttctgttcctgttcggttccagacactggtttcttgctttgttttccctggctaacagtctagtgcaaaagattaagattttatctgggggaaagaaaagaattttttaaaaaattaaactaaagatgttttaagctaaagcctgaatttgggatggaagcaggacagacaccgtggacagcgctgtatttacagacacacccagtgcgtgaagaccaacaaagtcacagtcgtatctctagaaagctctaaagaccatgttggaaagagtctccagttactgaacagatgaaaaggagcctgtgagagggctgttaacattagcaaatattttttccttgttttttctttgttaaaaccaaactggttcacctgaatcatgaattgagaagaaataattttcatttctaaattaagtcccttttagtttgatcagacagcttgaatcagcatctcttcttccctgtcagcctgactcttcccttcccctctctcattccccatactccctattttcattccttttttaaaaaataatataagctacagaaaccaggtaagccctttatttccttaaatgttttgccagccacttaccaattgctaagtattgaatttcagaaaaaaaaaatgcatttactggcaaggagaagagcaaagttaaggcttgataccaatcgagctaaggatacctgctttggaagcatgtttattctgttccccagcaactctggcctccaaaatgggagaaaacgccagtgtgtttaaattgatagcagatatcacgacagatttaacctctgccatgtgttttttattttgttttttagcagtgctgactaagccgaagttttgtaaggtacataaaatccaatttatatgtaaacaagcaataatttaagttgagaacttatgtgttttaattgtataatttttgtgaggtatacatattgtggaattgactcaaaaatgaggtacttcagtattaaattagatatcttcatagcaatgtctcctaaaggtgttttgtaaaggatatcaatgccttgattagacctaatttgtagacttaagactttttattttctaaaccttgtgattctgcttataagtcatttatctaatctatatgatatgcagccgctgtaggaaccaattcttgatttttatatgtttatattctttcttaatgaaccttagaaagactacatgttactaagcaggccacttttatggttgttttt

SEQ ID NO: 5 - DNA, 58 bp, Homo sapiens
cccagggtgaataacctcggggctctgtccctccaatcccagggtgaataacctcggg